(19) United States
(12) Patent Application Publication
(10) Pub. No.: US 2018/0064619 A1
(43) Pub. Date: Mar. 8, 2018

(54) METHOD FOR COSMETIC TREATMENT OF THE HAIR

(71) Applicant: L'OREAL, Paris (FR)

(72) Inventors: Julie BRUN, Asnières sur Seine (FR); Sandrine MAGGIO, Le Perreux-sur-Marne (FR); Géraldine FACK, Levallois (FR)

(21) Appl. No.: 15/559,985

(22) PCT Filed: Mar. 25, 2016
(86) PCT No.: PCT/EP2016/056720

§ 371 Date: Sep. 20, 2017

(30) Foreign Application Priority Data
Mar. 25, 2015 (FR) 1552484

(51) Int. Cl. A61K 8/41; A61K 8/898; A61K 8/81; A61K 8/58; A61K 8/37; A61K 8/34; A61Q 5/12; A61Q 5/02

(52) U.S. Cl. CPC A61K 8/416; A61K 8/898; A61K 8/817; A61K 8/585; A61K 8/8158; A61K 2800/884; A61K 8/37; A61K 8/342; A61Q 5/12; A61Q 5/02; A61K 8/8152

(57) ABSTRACT

The present invention relates to a cosmetic hair treatment process, especially for caring for and/or cleansing the hair, comprising:
a step (i) of applying to said hair a “first” cosmetic composition, comprising one or more cationic surfactants, one or more silicones and one or more non-silicone fatty substances, and then
a step (ii) of applying to said hair a “second” cosmetic composition, comprising one or more cationic surfactants, one or more cationic polymers with a cationic charge density of greater than or equal to 4 meq./g, and one or more organosilanes.

Said process makes it possible especially to obtain conditioning that is both immediate and long-lasting.

The present invention relates to a cosmetic hair treatment process, which consists in sequentially applying a cosmetic composition comprising a cationic surfactant, a silicone and a non-silicone fatty substance, followed by a cosmetic composition comprising a cationic surfactant, a particular cationic polymer and an organosilane.

Hair may be damaged or embrittled by the action of external atmospheric agents such as light and bad weather, or by mechanical or chemical treatments, such as brushing, combing, dyeing, bleaching, permanent-waving and/or relaxing.

To overcome these drawbacks, it is common practice to make use of hair treatments which can condition the hair. These haircare compositions may be conditioning shampoos or hair conditioners, which may be in the form of hair gels or lotions or more or less thick creams.

To improve the cosmetic properties of these compositions, it is known practice to introduce therein conditioning agents, which are intended mainly to repair or limit the harmful or undesirable effects brought about by the various treatments or attacking factors to which hair fibers are more or less repeatedly subjected.

With this aim, it has already been proposed to use, inter alia, organosilanes in cosmetic care compositions, to give hair satisfactory conditioning properties. Such compositions are described, for example, in patent applications FR 2910276, EP 2343042 and EP 2111848.

However, the care compositions described in the prior art afford conditioning and disentangling properties that are not sufficiently long-lasting. Specifically, these properties generally do not sufficiently resist washing and tend to diminish from the first shampoo wash. In other words, even though these haircare compositions make it possible to obtain suitable results during and just after their use (at T0), it was found that these effects had a tendency to disappear gradually with hair washes; it was thus necessary to regularly renew the application of hair treatments to maintain adequate conditioning effects.

There is thus a real need for a cosmetic hair treatment process which does not have the drawbacks mentioned above, and which is especially capable of affording conditioning and disentangling properties that are not only satisfactory immediately after the first application, but also persistent with respect to washing, for example persistent after at least three shampoo washes.

The aim of the present invention is to propose such a cosmetic hair treatment process, making it possible to obtain both immediate and persistent (or long-lasting) conditioning.

One subject of the present invention is thus a cosmetic hair treatment process, comprising:
a step (i) of applying to said hair a “first” cosmetic composition, comprising one or more cationic surfactants, one or more silicones and one or more non-silicone fatty substances, and then
a step (ii) of applying to said hair a “second” cosmetic composition, comprising one or more cationic surfactants, one or more cationic polymers with a cationic charge density of greater than or equal to 4 meq./g, and one or more organosilanes;

said process not comprising an intermediate rinsing step between said application steps (i) and (ii).

Implementation of the process according to the invention makes it possible especially to obtain the desired properties immediately on application, and in particular suppleness, smoothing, volume and a non-charged, natural feel, while at the same time conserving light and individualized hairs; this process most particularly gives the hair improved capacity for disentangling.

Moreover, the properties afforded by said process according to the invention satisfactorily withstand the various attacking factors to which hair may be subjected, such as light, bad weather, washing and perspiration. They are particularly persistent with respect to shampoo washing, especially after at least three shampoo washes.

In the present description, the expression “at least one” is equivalent to the expression “one or more” and can be substituted for said expression; the expression “between” is equivalent to the expression “ranging from” and can be substituted for said expression, and implies that the limits are included.

Process

The cosmetic treatment process according to the invention thus comprises a first step (step (i)) consisting in applying to the hair a cosmetic composition (which may be called the “first composition”) comprising one or more cationic surfactants, one or more silicones and one or more non-silicone fatty substances.

This first step may or may not be followed by a leave-on step, for example a leave-on time of from 1 to 30 minutes, preferably from 1 to 15 minutes, better still from 2 to 15 minutes. Preferably, said first step is followed by a step of leaving on the composition, preferably for 1 to 15 minutes.

However, this first step is not followed by a rinsing step, for example with water, before performing the second step described below. This means that the second composition below is applied directly to the hair, which still bears said first composition. There is no step of “removal” of said first composition before applying said second composition.

In other words, the application steps (i) and (ii) are performed without an intermediate rinsing step.

The cosmetic treatment process according to the invention comprises a second step (step (ii)) consisting in applying to the hair a cosmetic composition (which may be called the “second composition”) comprising one or more cationic surfactants, one or more cationic polymers with a cationic charge density of greater than or equal to 4 meq./g, and one or more organosilanes.

Said second step is performed after said first step above; there is no intermediate rinsing step between said first and second steps.

The term “rinsing” especially means a usual rinsing step, for example with cold or hot water, for example of 30-40° C., for example for some 1 to 10 minutes, especially 1 to 10 minutes; the water optionally comprising additional compounds.

Preferably, said second application step is performed immediately after said first application step, especially about 1 to 30 minutes after said first application step, i.e. after the optional leave-on time of said first composition applied in the first step.

This second step may or may not be followed by a leave-on step, for example a leave-on time of from 1 to 25 minutes; it may also be followed by a rinsing step, for example with water, and/or may be followed by a drying step, for example with hot air.

Preferably, said second step is followed by a step of leaving on the composition, preferably for 1 to 25 minutes, especially 1 to 20 minutes, and a rinsing step, and then a drying step.

The step of applying said first composition and the step of applying said second composition are performed successively, or sequentially, which means that the step of applying said first composition precedes the step of applying said second composition; intermediate steps, for example of leaving on and/or of rinsing, possibly being present between these two application steps.

In a particular embodiment of the invention, it is possible to envisage, prior to said first step of the process, a “hair washing” step, comprising the application to said hair of a washing composition, for example of shampoo type, preferably comprising one or more detergent surfactants; preferably, said prior washing step may be followed by a rinsing step, for example with water, before performing the process according to the invention.

First Composition

The cosmetic treatment process according to the invention thus comprises a first step consisting in applying to the hair a cosmetic composition known as the “first composition”, comprising one or more cationic surfactants, one or more silicones and one or more non-silicone fatty substances.

a/Cationic Surfactants

Said first cosmetic composition thus comprises one or more cationic surfactants, advantageously chosen from optionally polyoxyalkylenated primary, secondary or tertiary fatty amine salts, quaternary ammonium salts, and mixtures thereof.

As quaternary ammonium salts, mention may be made especially of:
the quaternary ammonium salts of formula (Ia):

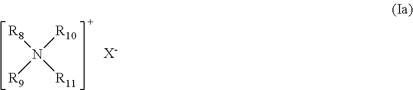

in which:

the groups R₈ to R₁₁, which may be identical or different, represent a linear or branched aliphatic group containing from 1 to 30 carbon atoms, or an aromatic group such as aryl or alkylaryl, at least one of the groups R₈ to R₁₁ containing from 8 to 30 and preferably from 12 to 24 carbon atoms, it being possible for the aliphatic groups to comprise heteroatoms such as, in particular, oxygen, nitrogen, sulfur and halogens; and
X⁻ is an anion chosen especially from the group of halides, phosphates, acetates, lactates, (C₁-C₄)alkyl sulfates, and (C₁-C₄)alkyl- or (C₁-C₄)alkylarylsulfonates.

The aliphatic groups R8 to R11 may be chosen from C₁-C₃₀ alkyl, C₁-C₃₀ alkoxy, (C₂-C₆) polyoxyalkylene, C₁-C₃₀ alkylamide, (C₁₂-C₂₂)alkylamido(C₂-C₆)alkyl, (C₁₂-C₂₂)alkyl acetate, and C₁-C₃₀ hydroxyalkyl groups.

Mention may be made especially of tetraalkylammonium halides, especially chlorides, such as dialkyldimethylammonium or alkyltrimethylammonium chlorides in which the alkyl group comprises from 12 to 22 carbon atoms, in particular behenyltrimethylammonium chloride, distearyldimethylammonium chloride, cetyltrimethylammonium chloride and benzyldimethylstearylammonium chloride.

Mention may also be made of palmitylamidopropyltrimethylammonium or stearamidopropyldimethyl-(myristyl acetate)-ammonium halides, and especially chlorides, especially the product sold under the name Ceraphyl® 70 by the company Van Dyk.
the quaternary ammonium salts of imidazoline of formula (IIa):

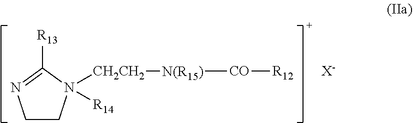

in which

R₁₂ represents an alkenyl or alkyl group comprising from 8 to 30 carbon atoms, for example derived from tallow fatty acids,

R₁₃ represents a hydrogen atom, a C₁-C₄ alkyl group or an alkenyl or alkyl group comprising from 8 to 30 carbon atoms,

R₁₄ represents a C₁-C₄ alkyl group,

R₁₅ represents a hydrogen atom or a C₁-C₄ alkyl group,

X⁻ is an anion chosen especially from the group of halides, phosphates, acetates, lactates, (C₁-C₄)alkyl sulfates, and (C₁-C₄)alkyl- or (C₁-C₄)alkylarylsulfonates.

Preferably, R₁₂ and R₁₃ denote a mixture of alkenyl or alkyl groups comprising from 12 to 21 carbon atoms, for example derived from tallow fatty acids, R₁₄ denotes a methyl group and R₁₅ denotes a hydrogen atom. Such a product is sold, for example, under the name Rewoquat® W75 or W90 by the company Evonik.
the di- or triquaternary ammonium salts of formula (IIIa):

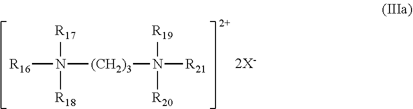

in which:
R₁₆ denotes an alkyl group comprising from 16 to 30 carbon atoms, which is optionally hydroxylated and/or optionally interrupted with one or more oxygen atoms,
R₁₇ denotes hydrogen, an alkyl group comprising from 1 to 4 carbon atoms or a group —(CH₂)₃—N⁺(R₁₆ₐ)(R₁₇ₐ)(R₁₈ₐ); R₁₆ₐ, R₁₇ₐ and R₁₈ₐ, which may be identical or different, denoting hydrogen or an alkyl group comprising from 1 to 4 carbon atoms,
R₁₈, R₁₉, R₂₀ and R₂₁, which may be identical or different, denote hydrogen or an alkyl group comprising from 1 to 4 carbon atoms, and
X⁻ is an anion, chosen especially from the group of halides, acetates, phosphates, nitrates, (C₁-C₄)alkyl sulfates and (C₁-C₄)alkyl- and (C₁-C₄)alkylarylsulfonates, in particular methyl sulfate and ethyl sulfate.

Such compounds are, for example, Finquat CT-P (Quaternium 89) and Finquat CT (Quaternium 75), sold by the company Finetex.
the quaternary ammonium salts containing one or more ester functions, of formula (IVa) below:

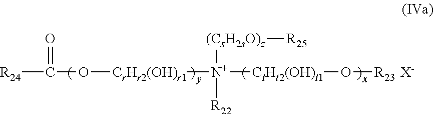

in which:
R₂₂ is chosen from C₁-C₆ alkyl groups and C₁-C₆ hydroxyalkyl or dihydroxyalkyl groups,
R₂₃ is chosen from the group R₂₆—C(═O)—; linear or branched, saturated or unsaturated C₁-C₂₂ hydrocarbon-based groups R₂₇; and a hydrogen atom,
R₂₅ is chosen from the group R₂₈—C(═O)—; linear or branched, saturated or unsaturated C₁-C₆ hydrocarbon-based groups R₂₉; and a hydrogen atom,
R₂₄, R₂₆ and R₂₈, which may be identical or different, are chosen from saturated or unsaturated, linear or branched C₇-C₂₁ hydrocarbon-based groups,
r, s and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10,
X⁻ is an anion,

it being understood that r2+r1=2r and t1+t2=2t, and that the sum x+y+z ranges from 1 to 15,

with the proviso that when x=0 then R₂₃ denotes R₂₇ and that when z=0 then R₂₅ denotes R₂₉.

The alkyl groups R₂₂ may be linear or branched, preferably linear. Preferably, R₂₂ denotes a methyl, ethyl, hydroxyethyl or dihydroxypropyl group, and more particularly a methyl or ethyl group.

Advantageously, the sum x+y+z ranges from 1 to 10.

When R₂₃ is a hydrocarbon-based group R₂₇, it may comprise from 12 to 22 carbon atoms, or else may comprise from 1 to 3 carbon atoms.

When R₂₅ is a hydrocarbon-based group R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based groups, and more particularly from linear or branched C₁₁-C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide, preferably chloride, bromide or iodide, a (C₁-C₄)alkyl sulfate, a (C₁-C₄)alkylsulfonate or a (C₁-C₄)alkylarylsulfonate, a methanesulfonate, a phosphate, a nitrate, a tosylate, an anion derived from an organic acid such as an acetate or a lactate or any other anion that is compatible with the ammonium bearing an ester function. The anion X⁻ is more particularly a chloride, a methyl sulfate or an ethyl sulfate.

Use is more particularly made, in the composition according to the invention, of the ammonium salts of formula (IVa) in which:
R₂₂ denotes a methyl or ethyl group,
x and y are equal to 1,
z is equal to 0 or 1,
r, s and t are equal to 2,
R₂₃ is chosen from the group R₂₆—C(═O)—, methyl, ethyl or C₁₄-C₂₂ hydrocarbon-based groups; and a hydrogen atom,
R₂₅ is chosen from the group R₂₈—C(═O)—; and a hydrogen atom,
R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Among the compounds of formula (IVa), mention may be made of salts, especially the chloride or methyl sulfate, of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably contain 14 to 18 carbon atoms and are obtained more particularly from a plant oil such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures especially of plant or animal origin, or by transesterification of the methyl esters thereof. This esterification may be followed by a quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin. Such compounds are sold, for example, under the names Dehyquart® by the company Henkel, Stepanquat® by the company Stepan, Noxamium® by the company CECA or Rewoquat® WE 18 by the company Evonik.

The composition according to the invention may contain, for example, a mixture of quaternary ammonium monoester, diester and triester salts with a majority by weight of diester salts. Use may also be made of the ammonium salts containing at least one ester functional group that are described in patents US-A-4 874 554 and US-A-4 137 180. Use may also be made of behenoylhydroxypropyltrimethylammonium chloride, for example, sold by the company Kao under the name Quartamin BTC 131.

Preferably, the ammonium salts containing at least one ester function contain two ester functions.

Preferably, the cationic surfactants are chosen from those of formula (Ia) or (IVa), and better still from cetyltrimethylammonium, behenyltrimethylammonium and dipalmitoylethylhydroxyethylmethylammonium salts and mixtures thereof; and more particularly from behenyltrimethylammonium chloride or methosulfate, cetyltrimethylammonium chloride or methosulfate, dipalmitoylethylhydroxyethylmethylammonium chloride or methosulfate, and mixtures thereof.

In a preferred embodiment, said first composition comprises at least two different cationic surfactants; especially at least one cationic surfactant chosen from those of formula (Ia) and at least one cationic surfactant chosen from those of formula (IVa).

Said first cosmetic composition comprises said cationic surfactant(s) in an amount ranging from 0.1% to 3.5% by weight, preferably from 0.2% to 3.5% by weight and preferentially from 0.3% to 3% by weight, relative to the total weight of the composition.

b/Silicones

Said first cosmetic composition according to the invention also comprises one or more silicones, which may be solid or liquid, volatile or non-volatile, and amino or non-amino.

As silicones that may be used, mention may be made, alone or as a mixture, of polydialkylsiloxanes and especially polydimethylsiloxanes (PDMSs), polydiarylsiloxanes, polyalkylarylsiloxanes, silicone gums and resins, and also organopolysiloxanes (or organomodified polysiloxanes, or alternatively organomodified silicones) which are polysiloxanes comprising in their structure one or more organofunctional groups, generally attached via a hydrocarbon-based group, and preferably chosen from aryl groups, amino groups, alkoxy groups and polyoxyethylene or polyoxypropylene groups.

The organomodified silicones may be polydiarylsiloxanes, especially polydiphenylsiloxanes, and polyalkylarylsiloxanes, functionalized with the organofunctional groups mentioned previously. The polyalkylarylsiloxanes are particularly chosen from linear and/or branched polydimethyl/methylphenylsiloxanes and polydimethyl/diphenylsiloxanes.

Among the organomodified silicones, mention may be made of organopolysiloxanes comprising:
polyethyleneoxy and/or polypropyleneoxy groups optionally comprising C₆-C₂₄ alkyl groups, such as dimethicone copolyols and especially those sold by the company Dow Corning under the name DC 1248 or the oils Silwet® L 722, L 7500, L 77 and L 711 from the company Union Carbide; or else (C₁₂)alkylmethicone copolyols and especially those sold by the company Dow Corning under the name Q2-5200;
substituted or unsubstituted amine groups, in particular C₁-C₄ aminoalkyl groups; mention may be made of the products sold under the names GP4 Silicone Fluid and GP7100 by the company Genesee, or under the names Q2-8220 and DC929 or DC939 by the company Dow Corning;
thiol groups, such as the products sold under the names GP 72 A and GP 71 from Genesee;
alkoxylated groups, such as the product sold under the name Silicone Copolymer F-755 by SWS Silicones and Abil Wax® 2428, 2434 and 2440 by the company Goldschmidt;
hydroxylated groups, for instance polyorganosiloxanes bearing a hydroxyalkyl function;
acyloxyalkyl groups, such as the polyorganosiloxanes described in patent US-A-4 957 732;
anionic groups of the carboxylic acid type, as described, for example, in EP 186 507, or of the alkylcarboxylic type, such as the product X-22-3701E from the company Shin-Etsu; or else of the 2-hydroxyalkylsulfonate or 2-hydroxyalkylthiosulfate type, such as the products sold by the company Goldschmidt under the names Abil® S201 and Abil® S255;
hydroxyacylamino groups, such as the polyorganosiloxanes described in patent application EP 342 834; mention may be made, for example, of the product Q2-8413 from the company Dow Corning.

The silicones may also be chosen from polydialkylsiloxanes, among which mention may be made mainly of polydimethylsiloxanes bearing trimethylsilyl end groups.

Among these polydialkylsiloxanes, mention may be made of the following commercial products:
the Silbione® oils of the 47 and 70 047 series or the Mirasil® oils sold by Rhodia, such as, for example, the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from the company Rhodia.

In this category of polydialkylsiloxanes, mention may also be made of the products sold under the names Abil Wax® 9800 and 9801 by the company Goldschmidt, which are poly(C1-C20)dialkylsiloxanes.

Products that may be used more particularly in accordance with the invention are mixtures such as:
the mixtures formed from a polydimethylsiloxane hydroxylated at the chain end, or dimethiconol (CTFA), and from a cyclic polydimethylsiloxane, also known as cyclomethicone (CTFA), such as the product Q2-1401 sold by the company Dow Corning.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, mention may be made of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Preferably, the composition according to the invention comprises one or more amino silicones. The term “amino silicone” denotes any silicone comprising at least one primary, secondary or tertiary amine or a quaternary ammonium group.

The weight-average molecular masses of these amino silicones may be measured by gel permeation chromatography (GPC) at room temperature (25° C.), as polystyrene equivalent. The columns used are μ styragel columns. The eluent is THF and the flow rate is 1 ml/min. 200 μl of a 0.5% by weight solution of silicone in THF are injected. Detection is performed by refractometry and UV-metry.

As amino silicone that may be used in the context of the invention, mention may be made of:

a) the polysiloxanes corresponding to formula (A):

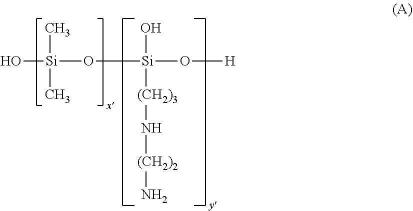

in which x′ and y′ are integers such that the weight-average molecular weight (Mw) is between 5000 and 500 000 approximately;

b) the amino silicones corresponding to formula (B):

R′ₐG₃₋ₐ-Si(OSiG₂)ₙ-(OSiG_{b}R′_{2-b})ₘ—O—SiG₃₋ₐ-R′ₐ (B)

in which:
G, which may be identical or different, denotes a hydrogen atom or a phenyl, OH or C₁-C₈ alkyl, for example methyl, or C₁-C₈ alkoxy, for example methoxy, group,
a, which may be identical or different, denotes 0 or an integer from 1 to 3, in particular 0,
b denotes 0 or 1, in particular 1,
m and n are numbers such that the sum (n+m) ranges from 1 to 2000 and in particular from 50 to 150, n possibly denoting a number from 0 to 1999 and in particular from 49 to 149, and m possibly denoting a number from 1 to 2000 and in particular from 1 to 10;
R′, which may be identical or different, denotes a monovalent radical of formula —C_{q}H_{2q}L in which q is a number ranging from 2 to 8 and L is an optionally quaternized amino group chosen from the following groups:

—N(R″)₂; —N⁺(R″)₃ A-; —NR″-Q-N(R″)₂ and —NR″-Q-N⁺(R″)₃ A-,

in which R″, which may be identical or different, denotes hydrogen, phenyl, benzyl, or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A- represents a cosmetically acceptable anion, in particular a halide such as fluoride, chloride, bromide or iodide.

A first group of amino silicones corresponding to formula (B) is represented by the silicones known as “trimethylsilyl amodimethicone”, corresponding to formula (C):

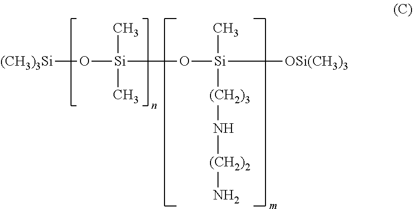

in which m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10.

A second group of amino silicones corresponding to formula (B) is represented by the silicones of formula (D) below:

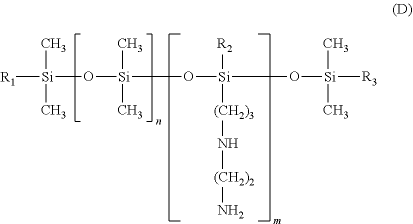

in which:
m and n are numbers such that the sum (n+m) ranges from 1 to 1000 and in particular from 50 to 250 and more particularly from 100 to 200; it being possible for n to denote a number from 0 to 999 and in particular from 49 to 249 and more particularly from 125 to 175, and for m to denote a number from 1 to 1000 and in particular from 1 to 10, and more particularly from 1 to 5;
R1, R2 and R3, which may be identical or different, represent a hydroxyl or C1-C4 alkoxy radical, at least one of the radicals R1 to R3 denoting an alkoxy radical.

Preferably, the alkoxy radical is a methoxy radical.

The hydroxy/alkoxy mole ratio ranges preferably from 0.2:1 to 0.4:1 and preferably from 0.25:1 to 0.35:1 and more particularly equals 0.3:1.

The weight-average molecular mass (Mw) of these silicones preferably ranges from 2000 to 1 000 000 and more particularly from 3500 to 200 000.

A third group of amino silicones corresponding to formula (B) is represented by the silicones of formula (E) below:

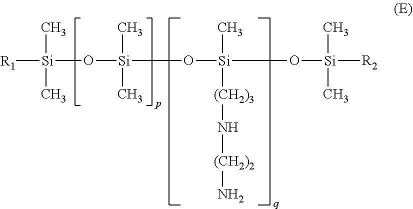

in which:
p and q are numbers such that the sum (p+q) ranges from 1 to 1000, in particular from 50 to 350 and more particularly from 150 to 250; it being possible for p to denote a number from 0 to 999 and in particular from 49 to 349 and more particularly from 159 to 239, and for q to denote a number from 1 to 1000, in particular from 1 to 10 and more particularly from 1 to 5;
R1 and R2, which are different, represent a hydroxyl or C1-C4 alkoxy radical, at least one of the radicals R1 or R2 denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly equals 1:0.95.

The weight-average molecular mass (Mw) of the silicone preferably ranges from 2000 to 200 000, even more particularly from 5000 to 100 000 and more particularly from 10 000 to 50 000.

The commercial products comprising silicones of structure (D) or (E) may include in their composition one or more other amino silicones of which the structure is different from formula (D) or (E).

A product containing amino silicones of structure (D) is sold by the company Wacker under the name Belsil® ADM 652.

A product containing amino silicones of structure (E) is sold by Wacker under the name Fluid WR 1300®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The numerical mean size of the silicone particles in the emulsion generally ranges from 3 nm to 500 nm. Preferably, in particular as amino silicones of formula (E), use is made of microemulsions of which the mean particle size ranges from 5 nm to 60 nm (limits included) and more particularly from 10 nm to 50 nm (limits included). Thus, use may be made according to the invention of the amino silicone microemulsions of formula (E) sold under the names Finish CT 96 E® or SLM 28020® by the company Wacker.

Another group of amino silicones corresponding to formula (B) is represented by the silicones of formula (F) below:

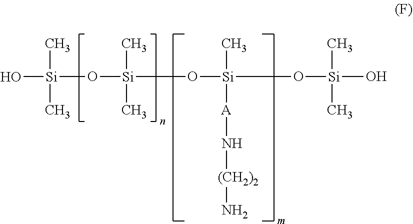

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms, this radical preferably being linear.

The weight-average molecular mass (Mw) of these amino silicones preferably ranges from 2000 to 1 000 000 and even more particularly from 3500 to 200 000.

A silicone corresponding to this formula is, for example, the Xiameter MEM 8299 Emulsion from Dow Corning.

Another group of amino silicones corresponding to formula (B) is represented by the silicones of formula (G) below:

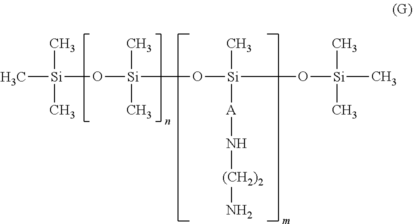

in which:
m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10;
A denotes a linear or branched alkylene radical containing from 4 to 8 carbon atoms and preferably 4 carbon atoms, This radical is preferably branched.

The weight-average molecular mass (Mw) of these amino silicones preferably ranges from 500 to 1 000 000 and even more particularly from 1000 to 200 000.

A silicone corresponding to this formula is, for example, DC2-8566 Amino Fluid from Dow Corning.

c) the amino silicones corresponding to formula (H):

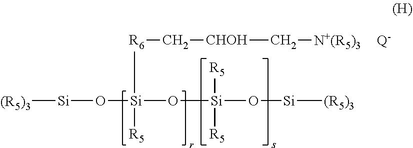

in which:
R₅ represents a monovalent hydrocarbon-based radical containing from 1 to 18 carbon atoms, and in particular a C₁-C₁₈ alkyl or C₂-C₁₈ alkenyl, for example methyl radical;
R₆ represents a divalent hydrocarbon-based radical, in particular a C₁-C₁₈ alkylene radical or a divalent C₁-C₁₈, for example C₁-C₈, alkyleneoxy radical linked to the Si via an SiC bond;
Q⁻ is an anion such as a halide, especially chloride, ion or an organic acid salt, especially acetate;
r represents a mean statistical value ranging from 2 to 20 and in particular from 2 to 8;
s represents a mean statistical value ranging from 20 to 200 and in particular from 20 to 50.

Such amino silicones are in particular described in U.S. Pat. No. 4,185,087.

d) the quaternary ammonium silicones of formula (I):

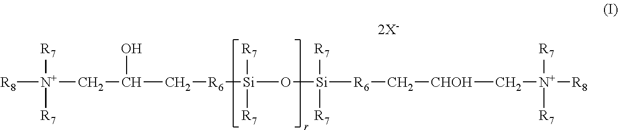

in which:
R₇, which may be identical or different, represent a monovalent hydrocarbon-based radical containing from 1 to 18 carbon atoms, and in particular a C₁-C₁₅ alkyl radical, a C₂-C₁₈ alkenyl radical or a ring comprising 5 or 6 carbon atoms, for example a methyl radical;
R₆ represents a divalent hydrocarbon-based radical, in particular a C₁-C₁₈ alkylene radical or a divalent C₁-C₁₈, for example C₁-C₈, alkyleneoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;
X⁻ is an anion such as a halide, especially chloride, ion or an organic acid salt, especially acetate;
r represents a mean statistical value ranging from 2 to 200 and in particular from 5 to 100.

These silicones are described, for example, in patent application EP-A 0 530 974.

e) the amino silicones of formula (J):

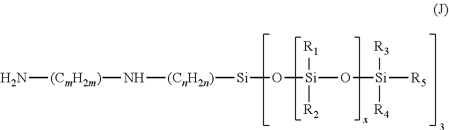

in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq./g.

f) the multiblock polyoxyalkylenated amino silicones, of the type (AB)n, A being a polysiloxane block and B being a polyoxyalkylene block comprising at least one amine group.

Said silicones preferably are constituted of repeating units of the following general formulae:

[—(SiMe₂O)ₓSiMe₂-R—N(R″)—R′—O(C₂H₄O)ₐ(C₃H₆O)_{b}—R′—N(H)—R—]

or alternatively

[—(SiMe₂O)ₓSiMe₂-R—N(R″)—R′—O(C₂H₄O)ₐ(C₃H₆O)_{b}—]

in which:
a is an integer greater than or equal to 1, preferably ranging from 5 to 200 and more particularly ranging from 10 to 100;
b is an integer between 0 and 200, preferably ranging from 4 to 100 and more particularly between 5 and 30;
x is an integer ranging from 1 to 10 000 and more particularly from 10 to 5000;
R″ is a hydrogen atom or a methyl;
R, which may be identical or different, represent a divalent linear or branched C2-C12 hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH(OH)CH₂— radical; preferentially R denotes a —CH₂CH₂CH₂OCH(OH)CH₂— radical;
R′, which may be identical or different, represent a linear or branched C2-C12 divalent hydrocarbon-based radical, optionally comprising one or more heteroatoms such as oxygen; preferably, R′ denotes an ethylene radical, a linear or branched propylene radical, a linear or branched butylene radical, or a —CH₂CH₂CH₂OCH(OH)CH₂— radical; preferentially, R′ denotes —CH(CH₃)—CH₂—.

The siloxane blocks preferably represent 50 mol % to 95 mol % of the total weight of the silicone, more particularly from 70 mol % to 85 mol %.

The amine content is preferably between 0.02 and 0.5 meq./g of copolymer in a 30% solution in dipropylene glycol, more particularly between 0.05 and 0.2.

The weight-average molecular mass (Mw) of the silicone is preferably between 5000 and 1 000 000 and more particularly between 10 000 and 200 000.

Mention may be made especially of the silicones sold under the names Silsoft A-843 or Silsoft A+ by Momentive.

The composition preferably comprises one or more silicones corresponding to formula (B), preferably to one of formulae (C), (D), (E), (F) and (G), and most particularly corresponding to formula (F).

Said first composition according to the invention comprises said silicone(s) in an amount ranging from 0.1% to 2% by weight, preferably from 0.2% to 1.7% by weight and preferentially from 0.3% to 1.6% by weight, relative to the total weight of the composition.

Most particularly, said first composition according to the invention comprises said amino silicone(s) in an amount ranging from 0.1% to 2% by weight, preferably from 0.2% to 1.7% by weight and preferentially from 0.3% to 1.6% by weight, relative to the total weight of the composition.

c/Non-Silicone Fatty Substances

The first composition according to the invention also comprises one or more non-silicone fatty substances, especially one or more solid non-silicone fatty substances.

The term “fatty substance” means an organic compound that is insoluble in water at room temperature (25° C.) and at atmospheric pressure (1 atm), i.e. which has a solubility of less than 5% by weight, preferably less than 1% by weight. They are generally soluble, under the same temperature and pressure conditions, in organic solvents such as chloroform, ethanol, benzene, liquid petroleum jelly or decamethylcyclopentasiloxane.

The term “non-silicone fatty substance” means a fatty substance whose structure does not comprise any silicon atoms, and which therefore especially does not comprise any siloxane groups. They generally bear in their structure a hydrocarbon-based chain comprising at least 6 carbon atoms. Advantageously, they are not oxyalkylenated and do not contain any —COOH functions.

The term “solid fatty substance” means a fatty substance that is solid at room temperature and atmospheric pressure (25° C., 1 atm); they preferably have a viscosity of greater than 2 Pa·s, measured at 25° C. and at a shear rate of 1 s⁻¹.

The solid non-silicone fatty substances that may be used in the context of the invention may be chosen from fatty alcohols, esters of a fatty acid and/or of a fatty alcohol, non-silicone waxes, ceramides, and mixtures thereof.

The term “fatty alcohol” means a long-chain aliphatic alcohol comprising from 8 to 40 carbon atoms and comprising at least one hydroxyl group OH. These fatty alcohols are neither oxyalkylenated nor glycerolated.

The solid fatty alcohols may be saturated or unsaturated, and linear or branched, and comprise from 8 to 40 carbon atoms. Preferably, the solid fatty alcohols have the structure R—OH with R denoting a linear alkyl group, optionally substituted with one or more hydroxyl groups, comprising from 8 to 40, better still from 10 to 30, or even from 12 to 24 and even better still from 14 to 22 carbon atoms.

The solid fatty alcohols that may be used are preferably chosen from saturated or unsaturated, linear or branched, preferably linear and saturated, (mono)alcohols comprising from 8 to 40 carbon atoms, better still from 10 to 30, or even from 12 to 24 atoms and better still from 14 to 22 carbon atoms.

The solid fatty alcohols that may be used may be chosen, alone or as a mixture, from:
lauryl alcohol (or 1-dodecanol);
myristyl alcohol (or 1-tetradecanol);
cetyl alcohol (or 1-hexadecanol);
stearyl alcohol (or 1-octadecanol);
arachidyl alcohol (or 1-eicosanol);
behenyl alcohol (or 1-docosanol);
lignoceryl alcohol (or 1-tetracosanol);
ceryl alcohol (or 1-hexacosanol);
montanyl alcohol (or 1-octacosanol);
myricyl alcohol (or 1-triacontanol).

Preferentially, the solid fatty alcohol is chosen from cetyl alcohol, stearyl alcohol, behenyl alcohol, myristyl alcohol, and mixtures thereof, such as cetylstearyl alcohol or cetearyl alcohol.

The solid esters of a fatty acid and/or of a fatty alcohol that may be used are preferably chosen from esters derived from a C₉-C₂₆ carboxylic fatty acid and/or from a C₉-C₂₆ fatty alcohol.

Preferably, these solid fatty esters are esters of a linear or branched, saturated carboxylic acid comprising at least 10 carbon atoms, preferably from 10 to 30 carbon atoms and more particularly from 12 to 24 carbon atoms, and of a linear or branched, saturated monoalkyl, comprising at least 10 carbon atoms, preferably from 10 to 30 carbon atoms and more particularly from 12 to 24 carbon atoms. The saturated carboxylic acids may be optionally hydroxylated, and are preferably monocarboxylic acids.

Esters of C₄-C₂₂ dicarboxylic or tricarboxylic acids and of C₁-C₂₂ alcohols and esters of mono-, di- or tricarboxylic acids and of di-, tri-, tetra- or pentahydroxy alcohols which are C₂-C₂₆ may also be used.

Mention may in particular be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C9-C26 alkyl palmitates, in particular myristyl, cetyl or stearyl palmitate; C9-C26 alkyl myristates, such as cetyl myristate, stearyl myristate and myristyl myristate; and C9-C26 alkyl stearates, in particular myristyl stearate, cetyl stearate and stearyl stearate; and mixtures thereof.

For the purposes of the present invention, a wax is a lipophilic compound, which is solid at room temperature (25° C.) and atmospheric pressure, with a reversible solid/liquid change of state, having a melting point greater than about 40° C., which may be up to 200° C., and having in the solid state an anisotropic crystal organization.

In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to room temperature, recrystallization of the wax, which is microscopically and macroscopically detectable (opalescence), is obtained.

In particular, the waxes that are suitable for use in the invention may be chosen from waxes of animal, plant or mineral origin, non-silicone synthetic waxes, and mixtures thereof.

Mention may be made especially of hydrocarbon-based waxes, for instance beeswax, especially of biological origin, lanolin wax and Chinese insect waxes; rice bran wax, carnauba wax, candelilla wax, ouricury wax, alfalfa wax, berry wax, shellac wax, Japan wax and sumach wax; montan wax, orange wax, lemon wax, microcrystalline waxes, paraffins and ozokerite; polyethylene waxes, the waxes obtained by Fisher-Tropsch synthesis and waxy copolymers, and also esters thereof.

Mention may thus be made of C₂ to C₆₀ microcrystalline waxes, such as Microwax HW.

Mention may also be made of the PM 500 polyethylene wax sold under the reference Permalen 50-L polyethylene.

Mention may also be made of waxes obtained by catalytic hydrogenation of animal or plant oils containing linear or branched C₈ to C₃₂ fatty chains. Among these waxes mention may especially be made of isomerized jojoba oil such as the trans-isomerized partially hydrogenated jojoba oil, especially the product manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate, especially the product sold under the name Hest 2T-4S® by the company Heterene.

The waxes obtained by hydrogenation of castor oil esterified with cetyl alcohol, such as those sold under the names Phytowax Castor 16L64® and 22L73® by the company Sophim, may also be used.

A wax that may be also used is a C₂₀ to C₄₀ alkyl (hydroxystearyloxy)stearate (the alkyl group containing from 20 to 40 carbon atoms), alone or as a mixture. Such a wax is especially sold under the names Kester Wax K 82 P®, Hydroxypolyester K 82 P® and Kester Wax K 80 P® by the company Koster Keunen.

It is also possible to use microwaxes in the compositions of the the invention; mention may be made especially of carnauba microwaxes, such as the product sold under the name MicroCare 350® by the company Micro Powders, synthetic-wax microwaxes, such as the product sold under the name MicroEase 114S® by the company Micro Powders, microwaxes constituted of a mixture of carnauba wax and polyethylene wax, such as the products sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

The waxes are preferably chosen from mineral waxes, for instance paraffin, petroleum jelly, lignite or ozokerite wax; plant waxes, for instance cocoa butter or cork fiber or sugar cane waxes, olive tree wax, rice wax, hydrogenated jojoba wax, ouricury wax, carnauba wax, candelilla wax, alfalfa wax, or absolute waxes of flowers, such as essential wax of blackcurrant flower sold by the company Bertin (France); waxes of animal origin, for instance beeswaxes or modified beeswaxes (cerabellina), spermaceti, lanolin wax and lanolin derivatives; microcrystalline waxes; and mixtures thereof.

The ceramides, or ceramide analogs such as glycoceramides, that may be used in the compositions according to the invention, are known; mention may in particular be made of ceramides of classes I, II, III and V according to the Dawning classification.

The ceramides or analogs thereof that may be used preferably correspond to the following formula:

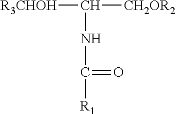

in which:
R₁ denotes a linear or branched, saturated or unsaturated alkyl group, derived from C₁₄-C₃₀ fatty acids, it being possible for this group to be substituted with a hydroxyl group in the alpha-position, or a hydroxyl group in the omega-position esterified with a saturated or unsaturated C₁₆-C₃₀ fatty acid;
R₂ denotes a hydrogen atom or a (glycosyl)n group, a (galactosyl)m group or a sulfogalactosyl group, in which n is an integer ranging from 1 to 4 and m is an integer ranging from 1 to 8;
R₃ denotes a C₁₅-C₂₆ hydrocarbon-based group, saturated or unsaturated in the alpha-position, it being possible for this group to be substituted with one or more C₁-C₁₄ alkyl groups;

it being understood that, in the case of natural ceramides or glycoceramides, R₃ may also denote a C₁₅-C₂₆ alpha-hydroxyalkyl group, the hydroxyl group being optionally esterified with a C₁₆-C₃₀ alpha-hydroxy acid.

The ceramides more particularly preferred are the compounds for which R₁ denotes a saturated or unsaturated alkyl derived from C₁₆-C₂₂ fatty acids; R₂ denotes a hydrogen atom and R₃ denotes a linear, saturated C₁₅ group.

Preferentially, ceramides are used for which R₁ denotes a saturated or unsaturated alkyl group derived from C₁₄-C₃₀ fatty acids; R₂ denotes a galactosyl or sulfogalactosyl group; and R₃ denotes a —CH═CH—(CH₂)₁₂—CH₃ group.

Use may also be made of the compounds for which R1 denotes a saturated or unsaturated alkyl radical derived from C12-C22 fatty acids; R2 denotes a galactosyl or sulfogalactosyl radical and R3 denotes a saturated or unsaturated C12-C22 hydrocarbon-based radical and preferably a —CH═CH—(CH₂)₁₂—CH₃ group.

As compounds that are particularly preferred, mention may also be made of 2-N-linoleoylaminooctadecane-1,3-diol, 2-N-oleoylaminooctadecane-1,3-diol, 2-N-palmitoylaminooctadecane-1,3-diol, 2-N-stearoylaminooctadecane-1,3-diol, 2-N-behenoylaminooctadecane-1,3-diol, 2-N-[2-hydroxypalmitoyl]aminooctadecane-1,3-diol; 2-N-stearoylaminooctadecane-1,3,4 triol and in particular N-stearoylphytosphingosine; 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof. N-oleoyldihydrosphingosine will preferably be used.

Preferably, said first composition according to the invention comprises said non-silicone fatty substance(s) in an amount ranging from 0.1% to 20% by weight, preferably from 0.5% to 15% by weight and preferentially from 1% to 10% by weight, relative to the total weight of the composition.

Most particularly, said first composition according to the invention comprises said solid non-silicone fatty substance(s) in an amount ranging from 0.5% to 15% by weight, preferably from 1% to 10% by weight and preferentially from 1.5% to 8% by weight, relative to the total weight of the composition.

d/Other Ingredients

Said first composition may also comprise water or a mixture of water and one or more cosmetically acceptable solvents chosen from C₁ to C₄ monoalcohols, such as ethanol, isopropanol, tert-butanol or n-butanol; polyols such as glycerol, propylene glycol and polyethylene glycols; and mixtures thereof.

Preferably, said first composition comprises water in a concentration preferably ranging from 50 to 99% by weight, especially from 60 to 98% by weight and better still from 70 to 97% by weight, relative to the total weight of said composition.

Said first composition may optionally also comprise one or more additional additives usually used in cosmetics.

The pH of the composition, if it is aqueous, is preferably between 3 and 7 and especially between 3 and 5.5.

Second Composition

The cosmetic treatment process according to the invention also comprises a second step consisting in applying to the hair a cosmetic composition (or second composition) comprising one or more cationic surfactants, one or more cationic polymers with a cationic charge density of greater than or equal to 4 meq./g, and one or more organosilanes.

a/Cationic Surfactants

Said second cosmetic composition thus comprises one or more cationic surfactants. Since the description of the cationic surfactants that may be used in said second composition is identical to the description given above, for the cationic surfactants that may be used in the first cosmetic composition, this description will not be repeated here.

Preferably, the cationic surfactants that may be used in the second composition according to the invention are chosen from those of formula (Ia) or (IVa), and better still from cetyltrimethylammonium, behenyltrimethylammonium and dipalmitoylethylhydroxyethylmethylammonium salts and mixtures thereof; and more particularly from behenyltrimethylammonium chloride or methosulfate, cetyltrimethylammonium chloride or methosulfate, dipalmitoylethylhydroxyethylmethylammonium chloride or methosulfate, and mixtures thereof.

Preferably, said second cosmetic composition comprises said cationic surfactant(s) in an amount ranging from 0.05% to 10% by weight, preferably from 0.1% to 5% by weight and preferentially from 0.3% to 3% by weight, relative to the total weight of the composition.

b/Organosilanes

Said second composition comprises one or more organosilanes, preferably chosen from compounds of formula (I) and/or oligomers thereof:

R₁Si(OR₂)_{z}(R₃)ₓ(OH)_{y} (I)

in which:
R₁ is a cyclic or acyclic, linear or branched, saturated or unsaturated C₁ to C₂₂, in particular C₂ to C₂₀, hydrocarbon-based chain, which may be substituted with a group chosen from amine groups NH₂ or NHR (R being a linear or branched C₁ to C₂₀, in particular C₁ to C₆, alkyl, a C₃ to C₄₀ cycloalkyl or a C₆ to C₃₀ aromatic radical); the hydroxyl group (OH); a thiol group; an aryl group (more particularly benzyl), which is possibly substituted with an NH₂ or NHR group; it being possible for R₁ to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3,
z denotes an integer ranging from 0 to 3, and
x denotes an integer ranging from 0 to 2,
with z+x+y=3.

The term “oligomer” means the polymerization products of the compounds of formula (I) comprising from 2 to 10 silicon atoms.

Preferably, R₁ is a linear or branched, preferably linear, saturated C₁ to C₂₂, in particular C₂ to C₁₂, chain, which may be substituted with an amine group NH₂ or NHR (R═C₁ to C₂₀, in particular C₁ to C₆, alkyl).

Preferably, R₂ represents an alkyl group comprising from 1 to 4 carbon atoms, better still a linear alkyl group comprising from 1 to 4 carbon atoms, and preferably an ethyl group.

Preferably, z ranges from 1 to 3.

Preferably, y=0.

Preferentially, z=3, and therefore x=y=0.

In one embodiment of the invention, the organosilane(s) are chosen from the compounds of formula (I) in which R₁ represents a linear alkyl group comprising from 7 to 18 carbon atoms and more particularly from 7 to 12 carbon atoms, or a C₁ to C₆, preferably C₂ to C₄, aminoalkyl group. More particularly, R₁ represents an octyl group.

In another embodiment of the invention, the organosilane(s) are chosen from the compounds of formula (I) in which R₁ is a linear or branched, saturated or unsaturated C₁ to C₂₂ hydrocarbon-based chain, substituted with an amine group NH₂ or NHR (with R═C₁ to C₂₀ alkyl, in particular C₁ to C₆, C₃ to C₄₀ cycloalkyl or C₆ to C₃₀ aromatic). In this variant, R₁ preferably represents a C₁ to C₆, and more preferably C₂ to C₄, aminoalkyl group.

Preferably, the second composition according to the invention comprises one or more organosilanes chosen from octyltriethoxysilane (OTES), dodecyltriethoxysilane, octadecyltriethoxysilane, hexadecyltriethoxysilane, 3-aminopropyltriethoxysilane (APTES), 2-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, N-(2-aminoethylaminomethyl)phenethyltrimethoxysilane, and oligomers and mixtures thereof; and more particularly chosen from octyltriethoxysilane (OTES) and 3-aminopropyltriethoxysilane (APTES), and oligomers and mixtures thereof.

The organosilanes used in the composition of the invention, especially those comprising a basic function, may be partially or totally neutralized in order to improve the water solubility thereof. In particular, the neutralizer may be chosen from organic or mineral acids, such as citric acid, tartaric acid, lactic acid or hydrochloric acid.

Preferably, the optionally neutralized organosilanes according to the invention are water-soluble and especially soluble at a concentration of 2% by weight, better still at a concentration of 5% by weight and even better still at a concentration of 10% by weight in water at a temperature of 25° C. and at atmospheric pressure (1 atm).

The term “soluble” indicates the formation of a single macroscopic phase.

Preferably, said second cosmetic composition comprises said organosilane(s) in an amount ranging from 0.1% to 15% by weight, preferably from 1% to 10% by weight and preferentially from 2% to 8% by weight, relative to the total weight of the composition.

c/Cationic Polymers of Particular Charge Density

Said second composition according to the invention also comprises one or more cationic polymers with a charge density of greater than or equal to 4 meq./g (milliequivalents per gram), preferably with a cationic charge density of greater than or equal to 5 meq./g; especially with a cationic charge density ranging from 4 to 20 meq./g, or even from 5 to 20 meq./g.

The cationic charge density of a polymer corresponds to the number of moles of cationic charges per unit mass of polymer under conditions in which it is totally ionized. It may be determined by calculation if the structure of the polymer is known, i.e. the structure of the monomers constituting the polymer and their molar proportion or weight proportion. It may also be determined experimentally by the Kjeldahl method.

The cationic polymers having a cationic charge density of greater than or equal to 4 meq./g that may be used in accordance with the present invention may be chosen from all those already known per se as improving the cosmetic properties of hair treated with detergent compositions, i.e. especially those described in patent application EP-A-0 337 354 and in French patent applications FR-A-2 270 846, 2 383 660, 2 598 611, 2 470 596 and 2 519 863.

In general, for the purposes of the present invention, the term “cationic polymer” denotes any polymer comprising cationic groups and/or groups that may be ionized into cationic groups.

The cationic polymers used in the present invention preferably have a number-average molecular weight (Mn) of greater than or equal to 50 000 g/mol, preferentially greater than or equal to 100 000 g/mol.

The cationic polymers that may be used according to the present invention are advantageously chosen from those that contain units comprising primary, secondary, tertiary and/or quaternary amine groups that may either form part of the main polymer chain or be borne by a side substituent directly connected thereto. More particularly, the cationic polymers may be chosen from polymers of the polyamine, polyaminoamide and quaternary polyammonium type, and polyalkyleneimines, and mixtures thereof.

Among the cationic polymers that may be used, mention may be made more particularly of:

(1) homopolymers or copolymers derived from acrylic or methacrylic esters or amides and comprising at least one of the units of the following formulae:

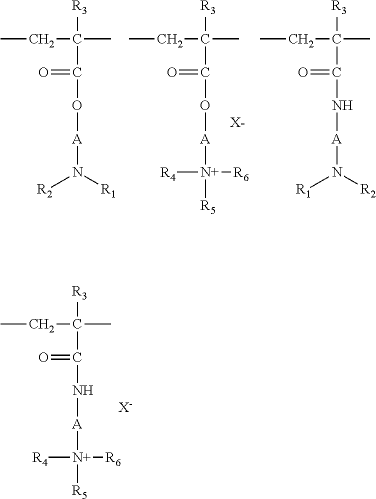

in which:
R3, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R4, R5 and R6, which may be identical or different, represent an alkyl group containing from 1 to 18 carbon atoms or a benzyl radical, preferably an alkyl group containing from 1 to 6 carbon atoms;
R1 and R2, which may be identical or different, represent a hydrogen atom or an alkyl group containing from 1 to 6 carbon atoms, preferably methyl or ethyl;
X denotes an anion derived from a mineral or organic acid, such as a methosulfate anion or a halide such as chloride or bromide.

The copolymers of family (1) may also contain one or more units derived from comonomers that may be chosen from the family of acrylamides, methacrylamides, diacetone acrylamides, acrylamides and methacrylamides substituted on the nitrogen with lower (C1-C4) alkyls, acrylic or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as that sold under the name Hercofloc by the company Hercules,
copolymers of acrylamide and of methacryloyloxyethyltrimethylammonium chloride, such as the products sold under the name Bina Quat P 100 by the company Ciba Geigy,
the copolymer of acrylamide and of methacryloyloxyethyltrimethylammonium methosulfate, such as that sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in detail in French patents 2 077 143 and 2 393 573;
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the copolymers sold under the name Styleze CC 10 by ISP,
quaternized vinylpyrrolidone/dimethylaminopropylmethacrylamide copolymers such as the product sold under the name Gafquat HS 100 by the company ISP,
preferably crosslinked polymers of methacryloyloxy(C1-C4)alkyltri(C1-C4)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, in particular methylenebisacrylamide.

Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba.

(2) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.

(3) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized.

(4) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical comprises from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.

(5) polymers obtained by reacting a polyalkylene polyamine comprising two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyamino amide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or else under the name PD 170 or Delsette 101 by the company Hercules in the case of the adipic acid/epoxypropyl/diethylenetriamine copolymer.

(6) alkyldiallylamine or dialkyldiallylammonium cyclopolymers, such as homopolymers or copolymers comprising, as the main chain constituent, units corresponding to formula (I) or (II):

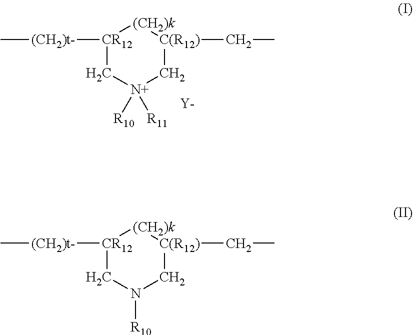

in which
k and t are equal to 0 or 1, the sum k+t being equal to 1;
R12 denotes a hydrogen atom or a methyl radical;
R10 and R11, independently of each other, denote a C1-C6 alkyl group, a C1-C5 hydroxyalkyl group, a C1-C4 amidoalkyl group; or alternatively R10 and R11 may denote, together with the nitrogen atom to which they are attached, a heterocyclic group such as piperidyl or morpholinyl; R10 and R11, independently of each other, preferably denote a C1-C4 alkyl group;
Y⁻ is an anion such as bromide, chloride, acetate, borate, citrate, tartrate, bisulfate, bisulfite, sulfate or phosphate.

Mention may be made more particularly of the homopolymer of dimethyldiallylammonium salts (for example chloride) for example sold under the name Merquat 100 by the company Nalco and the copolymers of diallyldimethylammonium salts (for example chloride) and of acrylamide, sold in particular under the name Merquat 550 or Merquat 7SPR.

(7) quaternary diammonium polymers comprising repeating units of formula:

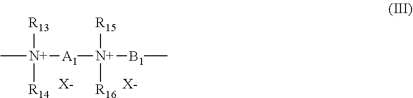

in which:
R13, R14, R15 and R16, which may be identical or different, represent aliphatic, alicyclic or arylaliphatic radicals comprising from 1 to 20 carbon atoms or C1-C12 hydroxyalkyl aliphatic radicals,

or else R13, R14, R15 and R16, together or separately, form, with the nitrogen atoms to which they are attached, heterocycles optionally comprising a second non-nitrogen heteroatom;

or else R13, R14, R15 and R16 represent a linear or branched C1-C6 alkyl radical substituted with a nitrile, ester, acyl, amide or —CO—O—R17-D or —CO—NH—R17-D group, where R17 is especially a C1-C6 alkylene and D is a quaternary ammonium group;
A1 and B1 represent linear or branched, saturated or unsaturated, divalent polymethylene groups comprising from 2 to 20 carbon atoms, which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;

it being understood that A1, R13 and R15 can form, with the two nitrogen atoms to which they are attached, a piperazine ring;

in addition, if A1 denotes a linear or branched, saturated or unsaturated alkylene or hydroxyalkylene radical, B1 may also denote a group (CH₂)ₙ—CO-D-OC—(CH₂)p- with n and p, which may be identical or different, being integers ranging from 2 to 20, and D denoting:
a) a glycol residue of formula —O—Z—O—, in which Z denotes a linear or branched hydrocarbon-based radical, or a group corresponding to one of the following formulae: —(CH₂CH₂O)x-CH₂CH₂— and —[CH₂CH(CH₃)O]y-CH₂CH(CH₃)—, in which x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula —NH—Y—NH—, in which Y denotes a linear or branched hydrocarbon-based radical, or else the divalent radical —CH₂—CH₂—S—S—CH₂—CH₂—;
d) a ureylene group of formula —NH—CO—NH—.

Preferably, X⁻ is an anion, such as chloride or bromide. These polymers have a number-average molar mass (Mn) generally of between 1000 and 100 000.

Mention may be made more particularly of polymers that are composed of repeating units corresponding to the formula:

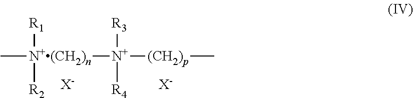

in which R1, R2, R3 and R4, which may be identical or different, denote an alkyl or hydroxyalkyl radical containing from 1 to 4 carbon atoms, n and p are integers ranging from 2 to 20, and X⁻ is an anion derived from a mineral or organic acid.

A particularly preferred compound of formula (IV) is the one for which R1, R2, R3 and R4 represent a methyl radical and n=3, p=6 and X═Cl, known as Hexadimethrine chloride according to the INCI (CTFA) nomenclature.

(8) polyguaternary ammonium polymers comprising units of formula (V):

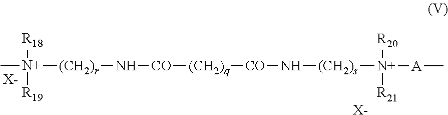

in which:
R18, R19, R20 and R21, which may be identical or different, represent a hydrogen atom or a methyl, ethyl, propyl, β-hydroxyethyl, β-hydroxypropyl or —CH₂CH₂(OCH₂CH₂)pOH radical, in which p is equal to 0 or to an integer between 1 and 6, with the proviso that R18 R19, R20 and R21 do not simultaneously represent a hydrogen atom,
r and s, which may be identical or different, are integers between 1 and 6,
q is equal to 0 or to an integer between 1 and 34,
X⁻ denotes an anion such as a halide,
A denotes a divalent dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by the company Miranol.

(9) quaternary polymers of vinylpyrrolidone and of vinylimidazole, for instance the products sold under the names Luviquat® FC 905, FC 550 and FC 370 by the company BASF.

(10) polymers comprising in their structure:

(a) one or more units corresponding to formula (A) below:

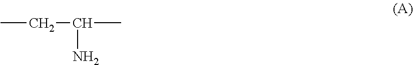

(b) optionally one or more units corresponding to formula (B) below:

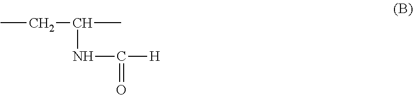

In other words, these polymers may be chosen in particular from homopolymers or copolymers comprising one or more units derived from vinylamine and optionally one or more units derived from vinylformamide.

Preferably, these cationic polymers are chosen from polymers comprising, in their structure, from 5 mol % to 100 mol % of units corresponding to the formula (A) and from 0 to 95 mol % of units corresponding to the formula (B), preferably from 10 mol % to 100 mol % of units corresponding to the formula (A) and from 0 to 90 mol % of units corresponding to the formula (B).

These polymers may be obtained, for example, by partial hydrolysis of polyvinylformamide. This hydrolysis may take place in acidic or basic medium.

The weight-average molecular mass of said polymer, measured by light scattering, may range from 1000 to 3 000 000 g/mol, preferably from 10 000 to 1 000 000 and more particularly from 100 000 to 500 000 g/mol.

The polymers comprising units of formula (A) and optionally units of formula (B) are sold in particular under the Lupamin name by the company BASF, for instance, in a non-limiting way, the products provided under the names Lupamin 9095, Lupamin 5095, Lupamin 1095, Lupamin 9030 (or Luviquat 9030) and Lupamin 9010.

Other cationic polymers that may be used in the context of the invention are polyalkyleneimines, in particular polyethyleneimines, polymers comprising vinylpyridine or vinylpyridinium units, condensates of polyamines and of epichlorohydrin, polyquaternary ureylenes and chitin derivatives.

Preferably, the cationic polymers are chosen from those of families (1), (6) and (7) mentioned above.

Use may preferably be made, alone or as a mixture, of:
alkyldiallylamine or dialkyldiallylammonium cyclopolymers, in particular homopolymers or copolymers of dimethyldiallylammonium salts (for example chloride), sold under the names Merquat 100, Merquat 550 and Merquat S by the company Nalco,
optionally crosslinked homopolymers or copolymers of methacryloyloxy(C1-C4)alkyltri(C1-C4)alkylammonium salts;
quaternary diammonium polymers comprising repeating units of formula (III);
homopolymers or copolymers derived from acrylic or methacrylic acids and comprising units below, as defined hereinabove:

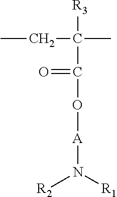

Better still, the cationic polymer(s) with a charge density of greater than or equal to 4 meq./g are chosen from 2-methacryloyloxyethyltrimethylammonium chloride (Polyquaternium-37), dimethyldiallylammonium chloride (Polyquaternium-6) and mixtures thereof.

Preferably, said second cosmetic composition comprises said cationic polymer(s) with a charge density of greater than or equal to 4 meq./g in an amount ranging from 0.01% to 15% by weight, preferably from 0.1% to 10% by weight and preferentially from 0.2% to 5% by weight, relative to the total weight of the composition.

d/Other Ingredients

Said second composition may also comprise water or a mixture of water and of one or more cosmetically acceptable solvents chosen from C₁ to C₄ monoalcohols, such as ethanol, isopropanol, tert-butanol or n-butanol; polyols such as glycerol, propylene glycol and polyethylene glycols; and mixtures thereof.

Preferably, said second composition comprises water in a concentration preferably ranging from 40 to 99% by weight, especially from 50 to 95% by weight and better still from 60 to 90% by weight, relative to the total weight of said composition.

The pH of the composition, if it is aqueous, is preferably between 3.5 and 7 and especially between 4 and 6.

Said second composition may optionally also comprise one or more liquid or solid, silicone or non-silicone fatty substances.

Preferably, the fatty substances that may be used in the composition according to the invention are non-silicone fatty substances.

Preferably, the fatty substance(s) are chosen from liquid or solid fatty alcohols; liquid or solid fatty esters; oils of mineral origin, plant oils of triglyceride type, and mixtures thereof.

The content of fatty substance(s), when they are present in the composition according to the invention, may range from 1% to 30% by weight, preferably from 5% to 25% by weight and more preferentially from 10% to 20% by weight relative to the total weight of the composition.

The second composition may optionally also comprise one or more organic acids, preferably chosen from saturated or unsaturated carboxylic acids; sulfonic acids; and mixtures thereof.

Preferably, the organic acid(s) are preferably chosen from carboxylic acids and especially from lactic acid, propanoic acid, butanoic acid, acetic acid, citric acid, maleic acid, glycolic acid, salicylic acid, malic acid and tartaric acid, and mixtures thereof, and more preferentially lactic acid.

The content of organic acid(s), when they are present in the composition according to the invention, may range from 0.1% to 10% by weight, and preferably from 0.5% to 5% by weight relative to the total weight of the composition.

Said second composition may optionally also comprise one or more additional additives usually used in cosmetics.

Optional Preliminary Composition

As mentioned above, in one particular embodiment of the invention, it is possible to envisage, prior to said first step of the process, a step of “washing” the hair, comprising the application to said hair of a washing composition, for example of shampoo type, preferably comprising one or more surfactants chosen especially from anionic surfactants and amphoteric surfactants, and mixtures thereof.

Preferably, said prior washing step may be followed by a rinsing step, for example with water, before performing the process according to the invention.

Said surfactants chosen especially from anionic surfactants and amphoteric surfactants, and mixtures thereof, may be present in the washing composition in a total content which may range from 5% to 35% by weight, preferably from 10% to 30% by weight and preferentially from 15% to 25% by weight, relative to the total weight of the composition.

Said anionic surfactants may be chosen from sulfate, sulfonate and/or carboxylic (or carboxylate) surfactants. Needless to say, a mixture of these surfactants may be used.

The carboxylic anionic surfactants that may be used thus comprise at least one carboxylic or carboxylate function (—COOH or —COO⁻).

They may be chosen from the following compounds: acylglycinates, acyllactylates, acylsarcosinates, acylglutamates; alkyl-D-galactosideuronic acids, alkyl ether carboxylic acids, alkyl(C6-30 aryl) ether carboxylic acids, alkylamido ether carboxylic acids; and also the salts of these compounds;

the alkyl and/or acyl groups of these compounds comprising from 6 to 30 carbon atoms, especially from 12 to 28, better still from 14 to 24 or even from 16 to 22 carbon atoms; the aryl group preferably denoting a phenyl or benzyl group;

these compounds possibly being polyoxyalkylenated, in particular polyoxyethylenated, and then preferably comprising from 1 to 50 ethylene oxide units and better still from 2 to 10 ethylene oxide units.

Use may also be made of the C6-C24 alkyl monoesters of polyglycoside-polycarboxylic acids, such as C6-C24 alkyl polyglycoside-citrates, C6-C24 alkyl polyglycoside-tartrates and C6-C24 alkyl polyglycoside-sulfosuccinates, and salts thereof.

Among the above carboxylic surfactants, mention may be made most particularly of polyoxyalkylenated alkyl(amido) ether carboxylic acids and salts thereof, in particular those comprising from 2 to 50 alkylene oxide and in particular ethylene oxide groups, such as the compounds sold by the company Kao under the Akypo names.

The polyoxyalkylenated alkyl(amido) ether carboxylic acids that may be used are preferably chosen from those of formula (1):

R₁—(OC₂H₄)ₙ—OCH₂COOA (1)

in which:
R1 represents a linear or branched C6-C24 alkyl or alkenyl radical, an alkyl(C8-C9)phenyl radical, a radical R2CONH—CH₂—CH₂— with R2 denoting a linear or branched C9-C21 alkyl or alkenyl radical,

preferably, R1 is a C8-C20 and preferably C8-C18 alkyl radical, and aryl preferably denotes phenyl,
n is an integer or decimal number (average value) ranging from 2 to 24 and preferably from 2 to 10,
A denotes H, ammonium, Na, K, Li, Mg or a monoethanolamine or triethanolamine residue.

It is also possible to use mixtures of compounds of formula (1), in particular mixtures of compounds containing different groups R1.

The polyoxyalkylenated alkyl(amido) ether carboxylic acids that are particularly preferred are those of formula (1) in which:
R1 denotes a C12-C14 alkyl, cocoyl, oleyl, nonylphenyl or octylphenyl radical,
A denotes a hydrogen or sodium atom, and
n ranges from 2 to 20, preferably from 2 to 10.

Even more preferentially, use is made of compounds of formula (1) in which R denotes a C12 alkyl radical, A denotes a hydrogen or sodium atom and n ranges from 2 to 10.

Preferentially, the carboxylic anionic surfactants are chosen, alone or as a mixture, from:
acylglutamates, especially of C6-C24 or even C12-C20, such as stearoylglutamates, and in particular disodium stearoylglutamate;
acylsarcosinates, especially of C6-C24 or even C12-C20, such as palmitoylsarcosinates, and in particular sodium palmitoylsarcosinate;
acyllactylates, especially of C12-C28 or even C14-C24, such as behenoyllactylates, and in particular sodium behenoyllactylate;
C6-C24 and in particular C12-C20 acylglycinates;
(C6-C24)alkyl ether carboxylates and especially (C12-C20)alkyl ether carboxylates;
polyoxyalkylenated (C₆-C₂₄)alkyl(amido) ether carboxylic acids, in particular those comprising from 2 to 50 ethylene oxide groups;

in particular in the form of alkali metal or alkaline-earth metal, ammonium or amino alcohol salts.

The sulfonate anionic surfactants that may be used comprise at least one sulfonate function (—SO₃H or —SO₃^{—}).

They may be chosen from the following compounds: alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds;

the alkyl groups of these compounds comprising from 6 to 30 carbon atoms, especially from 12 to 28, better still from 14 to 24 or even from 16 to 22 carbon atoms;

the aryl group preferably denoting a phenyl or benzyl group;

these compounds possibly being polyoxyalkylenated, in particular polyoxyethylenated, and then preferably comprising from 1 to 50 ethylene oxide units and better still from 2 to 10 ethylene oxide units.

Preferentially, the sulfonate anionic surfactants are chosen, alone or as a mixture, from:
C6-C24 and in particular C12-C20 alkylsulfosuccinates, in particular laurylsulfosuccinates;
C6-C24 and in particular C12-C20 alkyl ether sulfosuccinates;
(C6-C24)acylisethionates and preferably (C12-C18)acylisethionates,

in particular in the form of alkali metal or alkaline-earth metal, ammonium or amino alcohol salts.

The sulfate anionic surfactants that may be used comprise at least one sulfate function (—OSO₃H or —OSO₃⁻).

They may be chosen from the following compounds: alkyl sulfates, alkyl ether sulfates, alkylamido ether sulfates, alkylaryl polyether sulfates, monoglyceride sulfates; and also the salts of these compounds;

the alkyl groups of these compounds comprising from 6 to 30 carbon atoms, especially from 12 to 28, better still from 14 to 24 or even from 16 to 22 carbon atoms;

the aryl group preferably denoting a phenyl or benzyl group; these compounds possibly being polyoxyalkylenated, especially polyoxyethylenated, and then preferably comprising from 1 to 50 ethylene oxide units, better still from 2 to 10 ethylene oxide units.

Preferentially, the sulfate anionic surfactants are chosen, alone or as a mixture, from:
alkyl sulfates, in particular of C6-C24 or even C12-C20,
alkyl ether sulfates, in particular of C6-C24 or even C12-C20, preferably comprising from 2 to 20 ethylene oxide units;

in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferentially, the anionic surfactants are chosen, alone or as a mixture, from:
C6-C24 and in particular C12-C20 alkyl sulfates;
C6-C24 and in particular C12-C20 alkyl ether sulfates; preferably comprising from 2 to 20 ethylene oxide units;
C6-C24 and in particular C12-C20 alkylsulfosuccinates, in particular laurylsulfosuccinates;
C6-C24 and in particular C12-C20 alkyl ether sulfosuccinates;
(C6-C24)acylisethionates and preferably (C12-C18)acylisethionates,
C6-C24 and in particular C12-C20 acylsarcosinates; in particular palmitoylsarcosinates;
(C6-C24)alkyl ether carboxylates, preferably (C12-C20)alkyl ether carboxylates;
polyoxyalkylenated (C₆-C₂₄)alkyl(amido) ether carboxylic acids and salts thereof, in particular those comprising from 2 to 50 alkylene oxide and in particular ethylene oxide groups;
C6-C24 and in particular C12-C20 acylglutamates;
C6-C24 and in particular C12-C20 acylglycinates;

in particular in the form of alkali metal or alkaline-earth metal, ammonium or amino alcohol salts.

Said amphoteric surfactants may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C8-C20)alkylbetaines, sulfobetaines, (C₈-C₂₀)alkylsulfobetaines, (C8-C20)alkylamido(C1-C6)alkylbetaines, such as cocamidopropylbetaine, (C8-C20)alkylamido(C1-C6)alkylsulfobetaines, and also mixtures thereof.

Among the optionally quaternized secondary or tertiary aliphatic amine derivatives that may be used, mention may also be made of the products having the following respective structures (A1) and (A2):

Rₐ—CON(Z)CH_{2—}(CH₂)ₘ—N⁺(R_{b})(R_{c})(CH₂COO⁻) (A1)

in which:

Rₐ represents a C₁₀-C₃₀ alkyl or alkenyl group derived from an acid Rₐ—COOH preferably present in hydrolyzed coconut oil, or a heptyl, nonyl or undecyl group,

R_{b} represents a β-hydroxyethyl group,

R_{c} represents a carboxymethyl group,

m is equal to 0, 1 or 2,

Z represents a hydrogen atom or a hydroxyethyl or carboxymethyl group,

R_{a′}—CON(Z)CH_{2—}(CH₂)_{m′}—N(B)(B′) (A2)

in which:

B represents —CH₂CH₂OX′, with X′ representing —CH₂—COOH, CH₂—COOZ′, —CH₂CH₂—COOH, —CH₂CH₂—COOZ′, or a hydrogen atom,

B′ represents —(CH₂)_{z}—Y′, with z=1 or 2, and Y′ representing —COOH, —COOZ′, —CH₂—CHOH—SO₃H or —CH₂—CHOH—SO₃Z′,

m′ is equal to 0, 1 or 2,

Z represents a hydrogen atom or a hydroxyethyl or carboxymethyl group,

Z′ represents an ion derived from an alkali metal or alkaline-earth metal, such as sodium, potassium or magnesium; an ammonium ion; or an ion derived from an organic amine and especially from an amino alcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane.

R_{a′} represents a C₁₀-C₃₀ alkyl or alkenyl group of an acid R_{a′}COOH preferably present in hydrolyzed linseed oil or coconut oil, an alkyl group, especially a C₁₇ alkyl group, and its iso form, or an unsaturated C₁₇ group.

The compounds corresponding to formula (A2) are particularly preferred.

Among the compounds of formula (A2) for which X′ represents a hydrogen atom, mention may be made of the compounds known under the (CTFA) names sodium cocoamphoacetate, sodium lauroamphoacetate, sodium caproamphoacetate and sodium capryloamphoacetate.

Other compounds of formula (A2) are known under the (CTFA) names disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid and cocoamphodipropionic acid.

As examples of compounds of formula (A2), mention may be made of the cocoamphodiacetate sold by the company Rhodia under the trade name Miranol® C2M Concentrate, the sodium cocoamphoacetate sold under the trade name Miranol Ultra C 32 and the product sold by the company Chimex under the trade name Chimexane HA.

Use may also be made of compounds of formula (A3):

R_{a″}—NH—CH(Y″)—(CH₂)n-C(O)—NH—(CH₂)n′-N(R_{d})(Rₑ) (A3)

in which:
R_{a″} represents a C₁₀-C₃₀ alkyl or alkenyl group of an acid R_{a″}—C(O)OH, which is preferably present in hydrolyzed linseed oil or coconut oil;
Y″ represents the group —C(O)OH, —C(O)OZ″, —CH₂—CH(OH)—SO₃H or the group —CH₂—CH(OH)—SO₃—Z″, with Z″ representing a cation resulting from an alkali metal or alkaline-earth metal, such as sodium, an ammonium ion or an ion resulting from an organic amine;
R_{d} and Rₑ, independently of each other, represent a C1-C4 alkyl or hydroxyalkyl radical; and
n and n′, independently of each other, denote an integer ranging from 1 to 3.

Among the compounds of formula (A3), mention may in particular be made of the compound classified in the CTFA dictionary under the name sodium diethylaminopropyl cocoaspartamide and in particular the compound sold by the company Chimex under the name Chimexane HB.

Preferably, the amphoteric surfactants are chosen from (C8-C20)alkylbetaines, (C8-C20)alkylamido(C1-C6)alkylbetaines, (C8-C20)alkylamphoacetates and (C8-C20)alkylamphodiacetates, and mixtures thereof.

The washing composition may also comprise water or a mixture of water and of one or more cosmetically acceptable solvents chosen from C₁ to C₄ monoalcohols, such as ethanol, isopropanol, tert-butanol or n-butanol; polyols such as glycerol, propylene glycol and polyethylene glycols; and mixtures thereof.

Preferably, said washing composition comprises water in a concentration preferably ranging from 40 to 99% by weight, especially from 50 to 95% by weight and better still from 60 to 90% by weight, relative to the total weight of said composition.

In a preferred embodiment, the washing composition advantageously comprises one or more cationic polymers with a charge density of greater than or equal to 4 meq./g (milliequivalents per gram), preferably greater than or equal to 5 meq./g; especially with a cationic charge density ranging from 4 to 20 meq./g, or even from 5 to 20 meq./g.

Said cationic polymers are such as those already described above. Preferably, said cationic polymers are chosen from those of families (1), (6) and (7) mentioned above.

Use may preferably be made, alone or as a mixture, of:
alkyldiallylamine or dialkyldiallylammonium cyclopolymers, in particular homopolymers or copolymers of dimethyldiallylammonium salts (for example chloride), sold under the names Merquat 100, Merquat 550 and Merquat S by the company Nalco,
optionally crosslinked homopolymers or copolymers of methacryloyloxy(C1-C4)alkyltri(C1-C4)alkylammonium salts;
quaternary diammonium polymers comprising repeating units of formula (III),
homopolymers or copolymers derived from acrylic or methacrylic acids and comprising units below, as defined hereinabove:

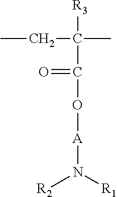

Better still, the cationic polymer(s) with a charge density of greater than or equal to 4 meq./g are chosen from 2-methacryloyloxyethyltrimethylammonium chloride (Polyquaternium-37), dimethyldiallylammonium chloride (Polyquaternium-6) and mixtures thereof.

Preferably, said washing composition comprises said cationic polymer(s) with a charge density of greater than or equal to 4 meq./g in an amount ranging from 0.01% to 15% by weight, preferably from 0.1% to 10% by weight and preferentially from 0.2% to 5% by weight, relative to the total weight of the composition.

In a particularly preferred variant of the invention, said washing composition comprises at least one cationic polymer with a charge density of greater than or equal to 4 meq./g identical to at least one cationic polymer with a charge density of greater than or equal to 4 meq./g present in the second cosmetic composition as described previously.

The cosmetic hair treatment process according to the invention finds a particularly advantageous application for the hygiene, cleansing, care and/or conditioning of the hair; advantageously for cleansing and conditioning the hair.

Preferably, it is a cosmetic process for cleansing and/or caring for and/or conditioning the hair.

In particular, it is a cosmetic process for cleansing and/or caring for and/or conditioning the hair, making it possible to obtain conditioning that is both immediate and long-lasting, i.e. persisting for at least three shampoo washes.

The present invention is illustrated in greater detail in the examples that follow (AM=active material).

### EXAMPLE 1

The first compositions below are prepared (weight %):

| | Composi- | Composi- | Composi- |
|---|---|---|---|
| | tion 1A | tion 1B | tion 1C |
| Mixture of myristyl alcohol, | 1.23% | 1.85% | 3.7% |
| cetyl alcohol and stearyl | | | |
| alcohol (2.5/95/2.5) | | | |
| Mixture of myristyl stearate | 0.27% | 0.4% | 0.8% |
| and myristyl palmitate | | | |
| Myristyl alcohol | 0.13% | 0.2% | 0.4% |
| Hydroxyethylcellulose | 1.2% | 1% | 0.25% |
| (MW 1 300 000) | | | |
| Dipalmitoylethylhydroxy- | 0.1% AM | 0.15% AM | 0.3% AM |
| ethylmethylammonium | | | |
| methosulfate (at 30% | | | |
| in cetearyl alcohol) | | | |
| Cetyltrimethylammonium | 0.2% AM | 0.32% AM | 0.62% |
| chloride | | | |
| Behenyltrimethylammonium | 0.16% AM | 0.24% AM | 0.47% AM |
| chloride | | | |
| Amino silicone (Xiameter | 0.5% AM | 0.8% AM | 1.5% AM |
| MEM-8299 Emulsion) | | | |
| Ceramide | 0.01% | 0.01% | 0.01% |
| Lactic or citric acid | 0.1% | 0.1% | 0.03% |
| Preserving agent, fragrance | qs | qs | qs |
| Water | qs 100% | qs 100% | qs 100% |

### EXAMPLE 2

The second composition below is prepared (weight %):

| | Composition 2 |
|---|---|
| Cetylstearyl alcohol (50/50 C₁₆/C₁₈) | 9% |
| Mixture of myristyl stearate and myristyl | 1% |
| palmitate | |
| White mineral oil | 3% |
| Dipalmitoylethylhydroxyethylmethyl- | 4.5% (1.35 AM + 3.15 AM) |
| ammonium methosulfate/cetearyl alcohol | |
| (30/70) | |
| Cetyltrimethylammonium chloride, | 3.2% (0.8 AM) |
| as an aqueous 25% solution | |
| 3-Aminopropyltriethoxysilane | 5% |
| Lactic acid | 2% |
| Polydimethyldiallylammonium chloride, | 1.2% (0.48 AM) |
| as an aqueous 40% solution (PQ6) | |
| Preserving agent, fragrance | qs |
| Water | qs 100% |

### EXAMPLE 3

The washing compositions below are prepared (weight %):

| | Composi- | Composi- | Composi- |
|---|---|---|---|
| | tion 3A | tion 3B | tion 3C |
| Sodium lauryl ether sulfate | 17.46% AM | 17.5% AM | 5.6% AM |
| Lauryl ether carboxylic | — | — | 4.16% AM |
| acid (5OE) | | | |
| Cocoyl betaine | 3% AM | 3% AM | 7.56% AM |
| Glycol distearate | 1.6% | 1.6% | — |
| Dimethicone | 2.7% AM | 0.75% AM | — |
| Amodimethicone | — | 1.15% AM | — |
| 2-Oleamido-1,3- | 0.01% | 0.01% | 0.01% |
| octadecanediol | | | |
| Carbomer | 0.15% | 0.15% | — |
| Polydimethyldiallyl- | 0.16% AM | 0.3% AM | 0.4% AM |
| ammonium chloride, | | | |
| as an aqueous 40% | | | |
| solution (PQ6) | | | |
| PPG-5-Ceteth-20 | 0.8% | 0.85% | 1% |
| Glycerol | — | — | 2% |
| NaCl | 0.73% | 0.73% | 1% |
| Preserving agent, fragrance | qs | qs | qs |
| pH agent | qs pH 5.5 | qs pH 5.5 | qs pH 5.5 |
| Water | qs 100% | qs 100% | qs 100% |

### EXAMPLE 4

The head of hair was washed beforehand with 12 g of composition 3A.

After rinsing, 12 g of the first cosmetic composition 1B were applied and left on for 5 minutes.

Without intermediate rinsing, 18 g of the second cosmetic composition 2 were then applied and left on for 5 minutes.

After rinsing, the wet hair is easy to disentangle, supple, smooth-feeling and the hairs are individualized.

During drying, the hair dries rapidly.

After drying, the hair is easy to disentangle, visually smooth, smooth-feeling, with a uniform feel from the root to the end.

The cosmetic performance qualities obtained persist after four shampoo washes, in particular in terms of suppleness, smoothness and disentangling, especially on dry hair.

### EXAMPLE 5

The following compositions are prepared (weight percentage of unmodified starting material)

Composition A:

| | Weight % |
|---|---|
| Cetylstearyl alcohol (50/50 C16/C18) | 6 |
| Mixture of myristyl stearate and myristyl palmitate | 1 |
| N-Oleyldihydrosphingosine | 0.1 |
| Dipalmitoylethylhydroxyethylmethylammonium | 4.5 |
| methosulfate at 30% in cetearyl alcohol | |
| 1,2-Octanediol | 0.3 |
| Citric acid monohydrate | 0.03 |
| Cetyl trimethylammonium chloride at 25% in water | 3.2 |
| White mineral oil | 2 |
| Polydimethylsiloxane containing aminoethyl aminopropyl | 1.7 |
| groups, containing methoxy and/or hydroxyl and alpha- | |
| omega silanol functions, as a cationic 60% aqueous | |
| emulsion | |
| Fragrance | qs |
| Water | qs 100% |

Composition B:

| | Weight % |
|---|---|
| Cetyltrimethylammonium chloride as an aqueous 25% | 0.25 |
| solution | |
| Hydroxyethylcellulose (MW: 1 300 000) | 0.25 |
| SMDI/polyethylene glycol copolymer bearing alkyl end | 3 |
| groups (methyl/C18) at 15% in a maltodextrin/water matrix | |
| Crosslinked ethyltrimethylammonium methacrylate chloride | 1 |
| homopolymer as a 50% inverse emulsion in mineral oil | |
| (PQ37) | |
| Fragrance | qs |
| Lactic acid | 1.6 |
| 3-Aminopropyltriethoxysilane | 5 |
| Cetylstearyl alcohol (50/50 C16/C18) | 4 |
| N-Oleyldihydrosphingosine | 0.01 |
| Water | qs 100% |

Composition Application Protocols

The compositions were applied to locks of sparingly sensitized hair (20% alkaline solubility, 20AS) at a rate of 1 g of composition per 2.7 g of hair.

| | Protocol 1 (invention) | Protocol 2 (comparative) |
|---|---|---|
| Step 1 | Apply composition A | Apply composition A |
| Step 2 | Leave on for 5 minutes | Leave on for 5 minutes |
| | Do not rinse | |
| Step 3 | | Rinse with water for 15 |
| | | seconds (water at 38° C., |
| | | flow rate: 3 liters/minute) |
| Step 4 | Apply composition B | Apply composition B |
| Step 5 | Leave on for 5 minutes | Leave on for 5 minutes |
| Step 6 | Rinse and dry | Rinse and dry |

The performance qualities in terms of smooth feel, lightness, suppleness, individualization and charged feel are then compared.

These performance qualities were evaluated on dry hair, by ten experts. The evaluation of the performance qualities is performed in a tactile (sensory) manner, by comparison of a lock treated according to protocol 1 with a lock treated according to protocol 2; the expert does not know which protocol was applied to the lock (blind test).

For each of the criteria, the ten experts declared which lock had the best level of performance qualities (“=” meaning that the performance is equivalent for the two locks).

The results obtained are given below:

| | Smooth | | Lightness | | Suppleness | |
|---|---|---|---|---|---|---|
| | Protocol | Protocol | Protocol | Protocol | Protocol | Protocol |
| | 2 | 1 | 2 | 1 | 2 | 1 |
| Expert 1 | − | + | − | + | − | + |
| Expert 2 | − | + | − | + | − | + |
| Expert 3 | − | + | − | + | − | + |
| Expert 4 | − | + | − | + | − | + |
| Expert 5 | − | + | − | + | − | + |
| Expert 6 | − | + | − | + | − | + |
| Expert 7 | − | + | + | − | − | + |
| Expert 8 | − | + | − | + | − | + |
| Expert 9 | − | + | + | − | − | + |
| Expert 10 | − | + | − | + | − | + |

| | | | |
|---|---|---|---|
| % of experts | 100% | 80% | 100% |
| in favor | | | |
| of P1 | | | |

| | Individualized | | Non-charged feel | |
|---|---|---|---|---|
| | Protocol 2 | Protocol 1 | Protocol 2 | Protocol 1 |
| Expert 1 | − | + | − | + |
| Expert 2 | − | + | − | + |
| Expert 3 | − | + | − | + |
| Expert 4 | − | + | − | + |
| Expert 5 | = | = | − | + |
| Expert 6 | − | + | − | + |
| Expert 7 | − | + | − | + |
| Expert 8 | − | + | − | + |
| Expert 9 | = | = | − | + |
| Expert 10 | − | + | − | + |

| | | |
|---|---|---|
| % of experts in | 80% | 100% |
| favor of P1 | | |

It is thus found that:
in 100% of the cases, the experts judged that the smooth feel, the suppleness and the non-charged feel of the locks which had undergone protocol P1 according to the invention were superior to those of the locks to which the comparative protocol P2 was applied;
in 80% of the cases, the experts judged that the lightness and the individualized nature of the locks of hair on which protocol P1 according to the invention had been applied were superior to those of the locks to which the comparative protocol P2 was applied.

1. A cosmetic hair treatment process, comprising:
a step (i) of applying to said hair a “first” cosmetic composition, comprising one or more cationic surfactants, one or more silicones and one or more non-silicone fatty substances, and then
a step (ii) of applying to said hair a “second” cosmetic composition, comprising one or more cationic surfactants, one or more cationic polymers with a cationic charge density of greater than or equal to 4 meq./g, and one or more organosilanes;
said process not comprising an intermediate rinsing step between said application steps (i) and (ii).

2. The process as claimed in claim 1, in which the application step (i) is followed by a step of leaving on the “first” cosmetic composition, preferably for 1 to 15 minutes.

3. The process as claimed in one of the preceding claims, in which the application step (ii) is followed by a step of leaving on the “second” cosmetic composition, preferably for 1 to 25 minutes, especially 1 to 20 minutes, and a rinsing step, and then optionally a drying step.

4. The process as claimed in one of the preceding claims, in which the “first” cosmetic composition comprises one or more cationic surfactants chosen from optionally polyoxyalkylenated primary, secondary or tertiary fatty amine salts, quaternary ammonium salts, and mixtures thereof; and especially chosen from:
the quaternary ammonium salts of formula (Ia): 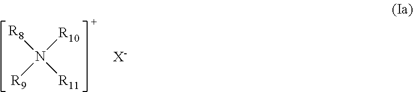
in which:
the groups R₈ to R₁₁, which may be identical or different, represent a linear or branched aliphatic group containing from 1 to 30 carbon atoms, or an aromatic group such as aryl or alkylaryl, at least one of the groups R₈ to R₁₁ containing from 8 to 30 and preferably from 12 to 24 carbon atoms, it being possible for the aliphatic groups to comprise heteroatoms such as, in particular, oxygen, nitrogen, sulfur and halogens; and
X⁻ is an anion chosen especially from the group of halides, phosphates, acetates, lactates, (C₁-C₄)alkyl sulfates, and (C₁-C₄)alkyl- or (C₁-C₄)alkylarylsulfonates;
the quaternary ammonium salts of imidazoline of formula (IIa): 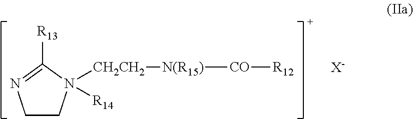
in which
R₁₂ represents an alkenyl or alkyl group comprising from 8 to 30 carbon atoms, for example derived from tallow fatty acids,
R₁₃ represents a hydrogen atom, a C₁-C₄ alkyl group or an alkenyl or alkyl group comprising from 8 to 30 carbon atoms,
R₁₄ represents a C₁-C₄ alkyl group,
R₁₅ represents a hydrogen atom or a C₁-C₄ alkyl group,
X⁻ is an anion chosen especially from the group of halides, phosphates, acetates, lactates, (C₁-C₄)alkyl sulfates, and (C₁-C₄)alkyl- or (C₁-C₄)alkylarylsulfonates,
the di- or triquaternary ammonium salts of formula (IIIa): 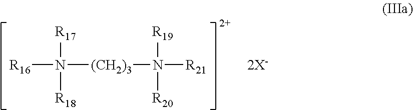
in which:
R₁₆ denotes an alkyl group comprising from 16 to 30 carbon atoms, which is optionally hydroxylated and/or optionally interrupted with one or more oxygen atoms,
R₁₇ denotes hydrogen, an alkyl group comprising from 1 to 4 carbon atoms or a group —(CH₂)₃—N⁺(R₁₆ₐ)(R₁₇ₐ)(R₁₈ₐ); R₁₆ₐ, R₁₇ₐ and R₁₈ₐ, which may be identical or different, denoting hydrogen or an alkyl group comprising from 1 to 4 carbon atoms,
R₁₈, R₁₉, R₂₀ and R₂₁, which may be identical or different, denote hydrogen or an alkyl group comprising from 1 to 4 carbon atoms, and
X⁻ is an anion, chosen especially from the group of halides, acetates, phosphates, nitrates, (C₁-C₄)alkyl sulfates and (C₁-C₄)alkyl- and (C₁-C₄)alkylarylsulfonates, in particular methyl sulfate and ethyl sulfate;
the quaternary ammonium salts containing one or more ester functions, of formula (IVa) below: 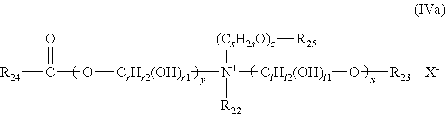
in which:
R₂₂ is chosen from C₁-C₆ alkyl groups and C₁-C₆ hydroxyalkyl or dihydroxyalkyl groups,
R₂₃ is chosen from the group R₂₆—C(═O)—; linear or branched, saturated or unsaturated C₁-C₂₂ hydrocarbon-based groups R₂₇; and a hydrogen atom,
R₂₅ is chosen from the group R₂₈—C(═O)—; linear or branched, saturated or unsaturated C₁-C₆ hydrocarbon-based groups R₂₉; and a hydrogen atom,
R₂₄, R₂₆ and R₂₈, which may be identical or different, are chosen from saturated or unsaturated, linear or branched C₇-C₂₁ hydrocarbon-based groups,
r, s and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10,
X⁻ is an anion,
it being understood that r2+r1=2r and t1+t2=2t, and that the sum x+y+z ranges from 1 to 15,
with the proviso that when x=0 then R₂₃ denotes R₂₇ and that when z=0 then R₂₅ denotes R₂₉.

5. The process as claimed in the preceding claim, in which the “first” cosmetic composition comprises one or more cationic surfactants chosen from the ammonium salts of formula (Ia) or (IVa), preferably of formula (IVa), in which:
R₂₂ denotes a methyl or ethyl group,
x and y are equal to 1,
z is equal to 0 or 1,
r, s and t are equal to 2,
R₂₃ is chosen from the group R₂₆—C(═O)—, methyl, ethyl or C₁₄-C₂₂ hydrocarbon-based groups; and a hydrogen atom,
R₂₅ is chosen from the group R₂₈—C(═O)—; and a hydrogen atom,
R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups;
and better still from cetyltrimethylammonium, behenyltrimethylammonium and dipalmitoylethylhydroxyethylmethylammonium salts and mixtures thereof; and more particularly from behenyltrimethylammonium chloride or methosulfate, cetyltrimethylammonium chloride or methosulfate, dipalmitoylethylhydroxyethylmethylammonium chloride or methosulfate, and mixtures thereof.

6. The process as claimed in one of the preceding claims, in which the “first” cosmetic composition comprises at least two different cationic surfactants; especially at least one cationic surfactant chosen from those of formula (Ia) and at least one cationic surfactant chosen from those of formula (IVa), the formulae being as defined in claim 4.

7. The process as claimed in one of the preceding claims, in which the “first” cosmetic composition comprises one or more cationic surfactant(s) in an amount ranging from 0.1% to 3.5% by weight, preferably from 0.2% to 3.5% by weight and preferentially from 0.3% to 3% by weight, relative to the total weight of the composition.

8. The process as claimed in one of the preceding claims, in which the “first” cosmetic composition comprises one or more amino silicones, chosen especially from:
a) the polysiloxanes corresponding to formula (A): 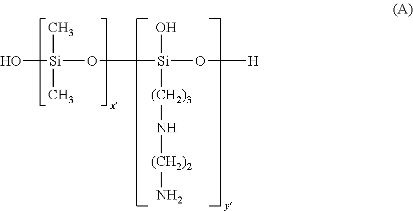
in which x′ and y′ are integers such that the weight-average molecular weight (Mw) is between 5000 and 500 000 approximately;
b) the amino silicones corresponding to formula (B):
R′ₐG₃₋ₐ-Si(OSiG₂)ₙ-(OSiG_{b}R′_{2-b})ₘ—O—SiG₃₋ₐ-R′ₐ (B)
in which:
G, which may be identical or different, denotes a hydrogen atom or a phenyl, OH or C₁-C₈ alkyl, for example methyl, or C₁-C₈ alkoxy, for example methoxy, group,
a, which may be identical or different, denotes 0 or an integer from 1 to 3, in particular 0,
b denotes 0 or 1, in particular 1,
m and n are numbers such that the sum (n+m) ranges from 1 to 2000 and in particular from 50 to 150, n possibly denoting a number from 0 to 1999 and in particular from 49 to 149, and m possibly denoting a number from 1 to 2000 and in particular from 1 to 10;
R′, which may be identical or different, denotes a monovalent radical of formula —C_{q}H_{2q}L in which q is a number ranging from 2 to 8 and L is an optionally quaternized amino group chosen from the following groups:
—N(R″)₂; —N⁺(R″)₃ A-; —NR″-Q-N(R″)₂ and —NR″-Q-N⁺(R″)₃ A-,
in which R″, which may be identical or different, denotes hydrogen, phenyl, benzyl, or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A- represents a cosmetically acceptable anion, in particular a halide such as fluoride, chloride, bromide or iodide;
c) the amino silicones corresponding to formula (H): 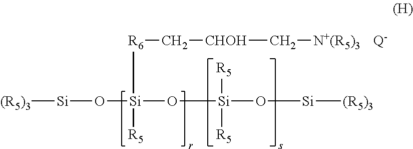
in which:
R₅ represents a monovalent hydrocarbon-based radical containing from 1 to 18 carbon atoms, and in particular a C₁-C₁₈ alkyl or C₂-C₁₈ alkenyl, for example methyl radical;
R₆ represents a divalent hydrocarbon-based radical, in particular a C₁-C₁₈ alkylene radical or a divalent C₁-C₁₈, for example C₁-C₈, alkyleneoxy radical linked to the Si via an SiC bond;
Q⁻ is an anion such as a halide, in particular chloride, ion or an organic acid salt, in particular acetate;
r represents a mean statistical value ranging from 2 to 20 and in particular from 2 to 8;
s represents a mean statistical value ranging from 20 to 200 and in particular from 20 to 50;
d) the quaternary ammonium silicones of formula (I): 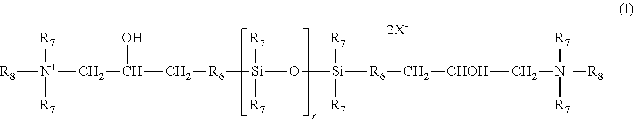
in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example a methyl radical;
R₆ represents a divalent hydrocarbon-based radical, in particular a C₁-C₁₈ alkylene radical or a divalent C₁-C₁₈, for example C₁-C₈, alkyleneoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;
X⁻ is an anion such as a halide, in particular chloride, ion or an organic acid salt, in particular acetate;
r represents a mean statistical value ranging from 2 to 200 and in particular from 5 to 100;
e) the amino silicones of formula (J): 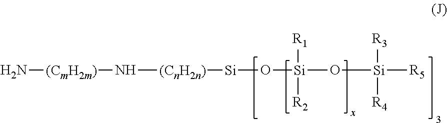
in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq./g;
f) the multiblock polyoxyalkylenated amino silicones, of the type (AB)n, A being a polysiloxane block and B being a polyoxyalkylene block comprising at least one amine group.

9. The process as claimed in one of the preceding claims, in which the “first” cosmetic composition comprises one or more amino silicones chosen from:
the “trimethylsilyl amodimethicone” silicones corresponding to formula (C): 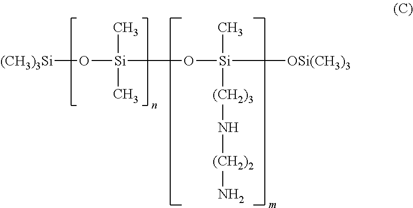
in which m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
the silicones of formula (D) below: 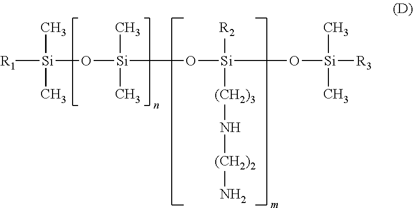
in which:
m and n are numbers such that the sum (n+m) ranges from 1 to 1000 and in particular from 50 to 250 and more particularly from 100 to 200; it being possible for n to denote a number from 0 to 999 and in particular from 49 to 249 and more particularly from 125 to 175, and form to denote a number from 1 to 1000 and in particular from 1 to 10, and more particularly from 1 to 5;
R1, R2 and R3, which may be identical or different, represent a hydroxyl or C1-C4 alkoxy radical, at least one of the radicals R1 to R3 denoting an alkoxy radical;
the silicones of formula (E) below: 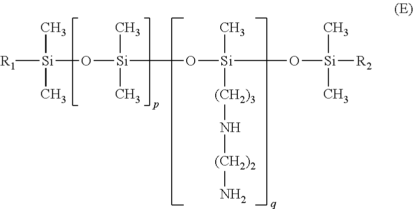
in which:
p and q are numbers such that the sum (p+q) ranges from 1 to 1000, in particular from 50 to 350 and more particularly from 150 to 250; it being possible for p to denote a number from 0 to 999 and in particular from 49 to 349 and more particularly from 159 to 239, and for q to denote a number from 1 to 1000, in particular from 1 to 10 and more particularly from 1 to 5;
R1 and R2, which are different, represent a hydroxyl or C1-C4 alkoxy radical, at least one of the radicals R1 or R2 denoting an alkoxy radical;
the silicones of formula (F) below: 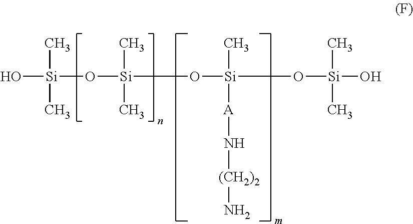
in which:
m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10;
A denotes a linear or branched alkylene radical containing from 4 to 8 carbon atoms and preferably 4 carbon atoms, this radical preferably being linear;
the silicones of formula (G) below: 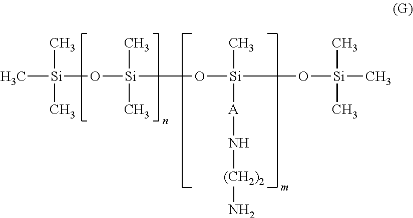
in which:
m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10;
A denotes a linear or branched alkylene radical containing from 4 to 8 carbon atoms and preferably 4 carbon atoms;
and most particularly corresponding to formula (F).

10. The process as claimed in one of the preceding claims, in which the “first” cosmetic composition comprises the silicone(s) in an amount ranging from 0.1% to 2% by weight, preferably from 0.2% to 1.7% by weight and preferentially from 0.3% to 1.6% by weight, relative to the total weight of the composition.

11. The process as claimed in one of the preceding claims, in which the “first” cosmetic composition comprises the amino silicone(s) in an amount ranging from 0.1% to 2% by weight, preferably from 0.2% to 1.7% by weight and preferentially from 0.3% to 1.6% by weight, relative to the total weight of the composition.

12. The process as claimed in one of the preceding claims, in which the “first” cosmetic composition comprises one or more solid non-silicone fatty substances, chosen especially from fatty alcohols, fatty acid and/or fatty alcohol esters, non-silicone waxes, ceramides, and mixtures thereof.

13. The process as claimed in one of the preceding claims, in which the “first” cosmetic composition comprises said non-silicone fatty substance(s) in an amount ranging from 0.1% to 20% by weight, preferably from 0.5% to 15% by weight and preferentially from 1% to 10% by weight, relative to the total weight of the composition.

14. The process as claimed in one of the preceding claims, in which the “first” cosmetic composition comprises said solid non-silicone fatty substance(s) in an amount ranging from 0.5% to 15% by weight, preferably from 1% to 10% by weight and preferentially from 1.5% to 8% by weight, relative to the total weight of the composition.

15. The process as claimed in one of the preceding claims, in which the “first” cosmetic composition comprises water in a concentration preferably ranging from 50% to 99% by weight, especially from 60% to 98% by weight and better still from 70% to 97% by weight, relative to the total weight of said composition.

16. The process as claimed in one of the preceding claims, in which the “second” cosmetic composition comprises one or more cationic surfactants chosen from optionally polyoxyalkylenated primary, secondary or tertiary fatty amine salts, quaternary ammonium salts, and mixtures thereof; and especially chosen from:
the quaternary ammonium salts of formula (Ia): 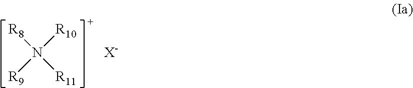
in which:
the groups R₈ to R₁₁, which may be identical or different, represent a linear or branched aliphatic group containing from 1 to 30 carbon atoms, or an aromatic group such as aryl or alkylaryl, at least one of the groups R₈ to R₁₁ containing from 8 to 30 and preferably from 12 to 24 carbon atoms, it being possible for the aliphatic groups to comprise heteroatoms such as, in particular, oxygen, nitrogen, sulfur and halogens; and
X⁻ is an anion chosen especially from the group of halides, phosphates, acetates, lactates, (C₁-C₄)alkyl sulfates, and (C₁-C₄)alkyl- or (C₁-C₄)alkylarylsulfonates;
the quaternary ammonium salts of imidazoline of formula (IIa): 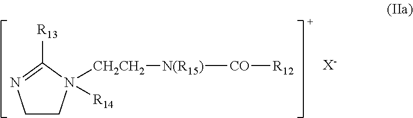
wherein
R₁₂ represents an alkenyl or alkyl group comprising from 8 to 30 carbon atoms, for example derived from tallow fatty acids,
R₁₃ represents a hydrogen atom, a C₁-C₄ alkyl group or an alkenyl or alkyl group comprising from 8 to 30 carbon atoms,
R₁₄ represents a C₁-C₄ alkyl group,
R₁₅ represents a hydrogen atom or a C₁-C₄ alkyl group,
X⁻ is an anion chosen especially from the group of halides, phosphates, acetates, lactates, (C₁-C₄)alkyl sulfates, and (C₁-C₄)alkyl- or (C₁-C₄)alkylarylsulfonates;
the di- or triquaternary ammonium salts of formula (IIIa): 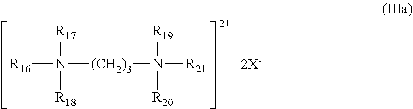
in which:
R₁₆ denotes an alkyl group comprising from 16 to 30 carbon atoms, which is optionally hydroxylated and/or optionally interrupted with one or more oxygen atoms,
R₁₇ denotes hydrogen, an alkyl group comprising from 1 to 4 carbon atoms or a group —(CH₂)₃—N⁺(R₁₆ₐ)(R₁₇ₐ)(R₁₈ₐ); R₁₆ₐ, R₁₇ₐ and R₁₈ₐ, which may be identical or different, denoting hydrogen or an alkyl group comprising from 1 to 4 carbon atoms,
R₁₈, R₁₉, R₂₀ and R₂₁, which may be identical or different, denote hydrogen or an alkyl group comprising from 1 to 4 carbon atoms, and
X⁻ is an anion, chosen especially from the group of halides, acetates, phosphates, nitrates, (C₁-C₄)alkyl sulfates and (C₁-C₄)alkyl- and (C₁-C₄)alkylarylsulfonates, in particular methyl sulfate and ethyl sulfate;
the quaternary ammonium salts containing one or more ester functions, of formula (IVa) below: 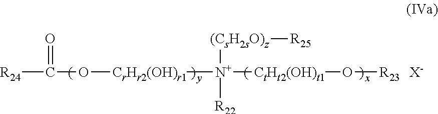
in which:
R₂₂ is chosen from C₁-C₆ alkyl groups and C₁-C₆ hydroxyalkyl or dihydroxyalkyl groups,
R₂₃ is chosen from the group R₂₆—C(═O)—; linear or branched, saturated or unsaturated C₁-C₂₂ hydrocarbon-based groups R₂₇; and a hydrogen atom,
R₂₅ is chosen from the group R₂₈—C(═O)—; linear or branched, saturated or unsaturated C₁-C₆ hydrocarbon-based groups R₂₉; and a hydrogen atom,
R₂₄, R₂₆ and R₂₈, which may be identical or different, are chosen from saturated or unsaturated, linear or branched C₇-C₂₁ hydrocarbon-based groups,
r, s and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10,
X⁻ is an anion,
it being understood that r2+r1=2r and t1+t2=2t, and that the sum x+y+z ranges from 1 to 15,
with the proviso that when x=0 then R₂₃ denotes R₂₇ and that when z=0 then R₂₅ denotes R₂₉.

17. The process as claimed in the preceding claim, in which the “second” cosmetic composition comprises one or more cationic surfactants chosen from the ammonium salts of formula (Ia) or (IVa), preferably of formula (IVa), in which:
R₂₂ denotes a methyl or ethyl group,
x and y are equal to 1,
z is equal to 0 or 1,
r, s and t are equal to 2,
R₂₃ is chosen from the group R₂₆—C(═O)—, methyl, ethyl or C₁₄-C₂₂ hydrocarbon-based groups; and a hydrogen atom,
R₂₅ is chosen from the group R₂₈—C(═O)—; and a hydrogen atom,
R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups;
and better still from cetyltrimethylammonium, behenyltrimethylammonium and dipalmitoylethylhydroxyethylmethylammonium salts and mixtures thereof; and more particularly from behenyltrimethylammonium chloride or methosulfate, cetyltrimethylammonium chloride or methosulfate, dipalmitoylethylhydroxyethylmethylammonium chloride or methosulfate, and mixtures thereof.

18. The process as claimed in one of the preceding claims, in which the “second” cosmetic composition comprises one or more cationic surfactant(s) in an amount ranging from 0.05% to 10% by weight, preferably from 0.1% to 5% by weight and preferentially from 0.3% to 3% by weight, relative to the total weight of the composition.

19. The process as claimed in one of the preceding claims, in which the “second” cosmetic composition comprises one or more organosilanes chosen from the compounds of formulae (I) and/or oligomers thereof:
R₁Si(OR₂)_{z}(R₃)ₓ(OH)_{y} (I)
in which:
R₁ is a cyclic or acyclic, linear or branched, saturated or unsaturated C₁ to C₂₂, in particular C₂ to C₂₀, hydrocarbon-based chain, which may be substituted with a group chosen from amine groups NH₂ or NHR (R being a linear or branched C₁ to C₂₀, in particular C₁ to C₆, alkyl, a C₃ to C₄₀ cycloalkyl or a C₆ to C₃₀ aromatic radical);
the hydroxyl group (OH); a thiol group; an aryl group (more particularly benzyl), which is possibly substituted with an NH₂ or NHR group; it being possible for R₁ to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3,
z denotes an integer ranging from 0 to 3, and
x denotes an integer ranging from 0 to 2,
with z+x+y=3.

20. The process as claimed in one of the preceding claims, in which the “second” cosmetic composition comprises said organosilane(s) in an amount ranging from 0.1% to 15% by weight, preferably from 1% to 10% by weight and preferentially from 2% to 8% by weight, relative to the total weight of the composition.

21. The process as claimed in one of the preceding claims, in which the “second” cosmetic composition comprises one or more cationic polymers chosen from:
(1) homopolymers or copolymers derived from acrylic or methacrylic esters or amides and comprising at least one of the units of the following formulae: 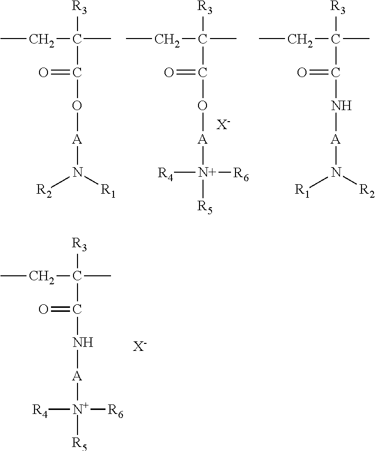
in which:
R3, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R4, R5 and R6, which may be identical or different, represent an alkyl group containing from 1 to 18 carbon atoms or a benzyl radical, preferably an alkyl group containing from 1 to 6 carbon atoms;
R1 and R2, which may be identical or different, represent a hydrogen atom or an alkyl group containing from 1 to 6 carbon atoms, preferably methyl or ethyl;
X denotes an anion derived from a mineral or organic acid, such as a methosulfate anion or a halide such as chloride or bromide;
(2) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers;
(3) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized;
(4) polyaminoamide derivatives resulting from the condensation of polyalkylene polyamines with polycarboxylic acids followed by alkylation with difunctional agents;
(5) polymers obtained by reacting a polyalkylene polyamine comprising two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyamino amide being reacted with epichlorohydrin in a mole ratio of epichlorohydrin relative to the secondary amine group of the polyaminoamide preferably of between 0.5:1 and 1.8:1;
(6) alkyldiallylamine or dialkyldiallylammonium cyclopolymers, such as homopolymers or copolymers comprising, as the main chain constituent, units corresponding to formula (I) or (II): 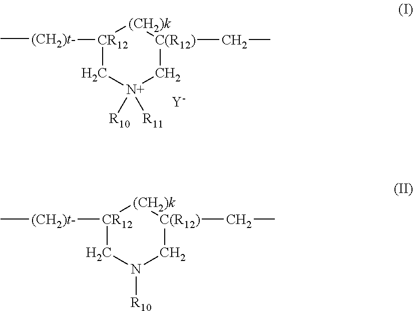
in which
k and t are equal to 0 or 1, the sum k+t being equal to 1;
R12 denotes a hydrogen atom or a methyl radical;
R10 and R11, independently of each other, denote a C1-C6 alkyl group, a C1-C5 hydroxyalkyl group, a C1-C4 amidoalkyl group; or alternatively R10 and R11 may denote, together with the nitrogen atom to which they are attached, a heterocyclic group such as piperidyl or morpholinyl; R10 and R11, independently of each other, preferably denote a C1-C4 alkyl group;
Y⁻ is an anion such as bromide, chloride, acetate, borate, citrate, tartrate, bisulfate, bisulfite, sulfate or phosphate;
(7) quaternary diammonium polymers comprising repeating units of formula: 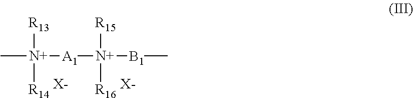
in which:
R13, R14, R15 and R16, which may be identical or different, represent aliphatic, alicyclic or arylaliphatic radicals comprising from 1 to 20 carbon atoms or C1-C12 hydroxyalkyl aliphatic radicals,
or else R13, R14, R15 and R16, together or separately, form, with the nitrogen atoms to which they are attached, heterocycles optionally comprising a second non-nitrogen heteroatom;
or else R13, R14, R15 and R16 represent a linear or branched C1-C6 alkyl radical substituted with a nitrile, ester, acyl, amide or —CO—O—R17-D or —CO—NH—R17-D group, where R17 is an alkylene and D is a quaternary ammonium group;
A1 and B1 represent linear or branched, saturated or unsaturated, divalent polymethylene groups comprising from 2 to 20 carbon atoms, which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;
it being understood that A1, R13 and R15 can form, with the two nitrogen atoms to which they are attached, a piperazine ring;
in addition, if A1 denotes a linear or branched, saturated or unsaturated alkylene or hydroxyalkylene radical, B1 may also denote a group (CH₂)n-CO-D-OC—(CH₂)p- with n and p, which may be identical or different, being integers ranging from 2 to 20, and D denoting:
a) a glycol residue of formula —O—Z—O—, in which Z denotes a linear or branched hydrocarbon-based radical, or a group corresponding to one of the following formulae: —(CH₂CH₂O)x-CH₂CH₂— and —[CH₂CH(CH₃)O]y-CH₂CH(CH₃)—, in which x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula —NH—Y—NH—, in which Y denotes a linear or branched hydrocarbon-based radical, or else the divalent radical —CH₂—CH₂—S—S—CH₂—CH₂—;
d) a ureylene group of formula —NH—CO—NH—.
Preferably, X⁻ is an anion, such as chloride or bromide. These polymers have a number-average molar mass (Mn) generally of between 1000 and 100 000;
(8) polyquaternary ammonium polymers comprising units of formula (V): 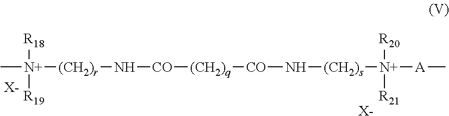
in which:
R18, R19, R20 and R21, which may be identical or different, represent a hydrogen atom or a methyl, ethyl, propyl, β-hydroxyethyl, β-hydroxypropyl or —CH₂CH₂(OCH₂CH₂)pOH radical, in which p is equal to 0 or to an integer between 1 and 6, with the proviso that R18 R19, R20 and R21 do not simultaneously represent a hydrogen atom,
r and s, which may be identical or different, are integers between 1 and 6,
q is equal to 0 or to an integer between 1 and 34,
X⁻ denotes an anion such as a halide,
A denotes a divalent dihalide radical or preferably represents —CH₂—CH₂—O—CH₂—CH₂—;
(9) quaternary polymers of vinylpyrrolidone and of vinylimidazole;
(10) polymers comprising in their structure:
(a) one or more units corresponding to formula (A) below: 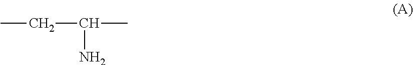
(b) optionally one or more units corresponding to formula (B) below: 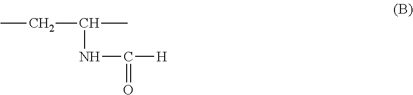
preferably chosen from those of families (1), (6) and (7).

22. The process as claimed in one of the preceding claims, in which the “second” cosmetic composition comprises one or more cationic polymers chosen from 2-methacryloyloxyethyltrimethylammonium chloride (Polyquaternium-37), dimethyldiallylammonium chloride (Polyquaternium-6) and mixtures thereof.

23. The process as claimed in one of the preceding claims, in which the “second” cosmetic composition comprises one or more cationic polymers with a charge density of greater than or equal to 4 meq./g, in an amount ranging from 0.01% to 15% by weight, preferably from 0.1% to 10% by weight and preferentially from 0.2% to 5% by weight, relative to the total weight of the composition.

24. The process as claimed in one of the preceding claims, in which the “second” cosmetic composition comprises water in a concentration preferably ranging from 40% to 99% by weight, especially from 50% to 95% by weight and better still from 60% to 90% by weight, relative to the total weight of said composition.

25. The process as claimed in one of the preceding claims, in which the “second” cosmetic composition comprises one or more organic acids, preferably chosen from saturated or unsaturated carboxylic acids; sulfonic acids; and mixtures thereof.

26. The process as claimed in one of the preceding claims, comprising, prior to said step (i) of applying a “first” cosmetic composition to said hair, a step of “washing” the hair, comprising the application to said hair of a washing composition, preferably comprising one or more detergent surfactants; preferably, said preliminary washing step being followed by a rinsing step, for example with water.

27. The process as claimed in claim 26, in which the washing composition comprises one or more surfactants chosen especially from anionic surfactants and amphoteric surfactants, and mixtures thereof, preferably present in a total content which may range from 5% to 35% by weight, preferably from 10% to 30% by weight and preferentially from 15% to 25% by weight, relative to the total weight of the composition.

28. The process as claimed in either of claims 26 and 27, in which the washing composition comprises one or more cationic polymers with a charge density of greater than or equal to 4 meq./g, preferably greater than or equal to 5 meq./g; especially with a cationic charge density ranging from 4 to 20 meq./g, or even from 5 to 20 meq./g; preferably, said cationic polymers being chosen from those of families (1), (6) and (7) above in claim 21; better still chosen from 2-methacryloyloxyethyltrimethylammonium chloride (Polyquaternium-37), dimethyldiallylammonium chloride (Polyquaternium-6) and mixtures thereof.

29. The process as claimed in one of claims 26 to 28, in which the washing composition comprises one or more cationic polymer(s) with a charge density of greater than or equal to 4 meq./g in an amount ranging from 0.01% to 15% by weight, preferably from 0.1% to 10% by weight and preferentially from 0.2% to 5% by weight, relative to the total weight of said composition.

30. The process as claimed in one of claims 26 to 29, in which the washing composition comprises at least one cationic polymer with a charge density of greater than or equal to 4 meq./g identical to at least one cationic polymer with a charge density of greater than or equal to 4 meq./g present in the second cosmetic composition.

31. The process as claimed in one of the preceding claims, for cleansing and/or caring for and/or conditioning the hair, especially for obtaining conditioning that is both immediate and long-lasting.
